(19) United States
(12) Patent Application Publication
(10) Pub. No.: US 2024/0260811 A1
(43) Pub. Date: Aug. 8, 2024

(54) MEDICAL SYSTEM AND NOTIFICATION METHOD

(71) Applicant: OLYMPUS CORPORATION, Tokyo (JP)

(72) Inventors: Masahiro FUJII, Tokyo (JP); Tetsuri KUWAZURU, Tokyo (JP); Shoei TSURUTA, Tokyo (JP); Kantaro NISHIKAWA, Tokyo (JP); Takeshi ARAI, Tokyo (JP)

(73) Assignee: OLYMPUS CORPORATION, Tokyo (JP)

(21) Appl. No.: 18/623,170

(22) Filed: Apr. 1, 2024

Related U.S. Application Data
(63) Continuation of application No. PCT/JP2023/003488, filed on Feb. 3, 2023.
(60) Provisional application No. 63/306,571, filed on Feb. 4, 2022.

(51) Int. Cl. A61B 1/00; G06T 7/00; G06T 7/11; G06T 7/90

(52) U.S. Cl. CPC A61B 1/00009; A61B 1/00055; G06T 7/0012; G06T 7/11; G06T 7/90; G06T 2207/10068

(57) ABSTRACT

A medical system includes a treatment tool provided with an end effector that treats a tissue, an imager capturing an image of the tissue, and a processor. The processor acquires color information of a target tissue from the captured image output from the imager. The target tissue is a portion of the tissue that covers at least a portion of the end effector that has slid into the tissue. The processor estimates a force applied to the target tissue by the end effector from the color information of the target tissue. The processor notifies an operator of a result of the estimation of the force.

### CROSS REFERENCE TO RELATED APPLICATIONS

This application is a continuation of International Patent Application No. PCT/JP2023/003488, having an international filing date of Feb. 3, 2023, which designated the United States, the entirety of which is incorporated herein by reference. U.S. Provisional Patent Application No. 63/306,571 filed on Feb. 4, 2022 is also incorporated herein by reference in its entirety.

### BACKGROUND OF THE INVENTION

In a treatment using an endoscope, when incision is performed while a tissue is lifted by a hook-knife type monopolar device, an operation of hooking the tissue with a knife to pull the tissue is performed. By applying energy while pulling the tissue with an appropriate tension, it is possible to shorten time to apply energy and narrow a range of thermal invasion, which allows to perform surgery safely and efficiently.

In addition, known is a method of displaying support information on a monitor based on information acquired from an endoscope image. For example, Japanese Unexamined Patent Application Publication No. 2014-213094 discloses a lesion evaluation information generator that calculates an evaluation value for evaluating a severity of a lesion based on a hue value and a saturation value of each of pixels included in an endoscopic image, and that includes a display means that displays the evaluation value on a predetermined display screen.

### SUMMARY OF THE INVENTION

In accordance with one of some embodiments, there is provided a medical system comprising:
a treatment tool provided with an end effector that treats a tissue;
an imager capturing an image of the tissue;
a processor; and
a memory including a program,
the memory and the program being configured to, together with the processor, cause the medical system to:
acquire color information of a target tissue from the captured image output from the imager, the target tissue being a portion of the tissue that covers at least a portion of the end effector that has slid into the tissue;
estimate a force applied to the target tissue by the end effector from the color information of the target tissue; and
notify an operator of a result of the estimation of the force.

In accordance with one of some embodiments, there is provided a medical system comprising:
a treatment tool provided with an end effector that treats a tissue;
a generator that adjusts energy output from the end effector;
an imager capturing an image of the tissue;
a processor; and
a memory including a program,
the memory and the program being configured to, together with the processor, cause the medical system to:
perform first processing of estimating a force applied to the tissue to be treated by the treatment tool from the image captured by the imager and changing an output setting of the generator based on a result of the estimation of the force;
perform second processing of detecting a metal in a periphery of the end effector from the image captured by the imager and changing the output setting of the generator based on a result of the detection of the metal, or
perform third processing of outputting a recommended output setting of the generator, accepting operation input to approve or reject the recommended output setting, and, in a case where the recommended output setting is approved or not rejected in the operation input, changing the output setting of the generator to the recommended output setting; and
wherein, in a case where the first processing or the third processing is performed,
the memory and the program, together with the processor, cause the medical system to:
acquire color information of a target tissue from the captured image output from the imager, the target tissue being a portion of the tissue that covers at least a portion of the end effector that has slid into the tissue;
estimate the force applied to the target tissue by the end effector from the color information of the target tissue; and
change the output setting of the generator based on a result of the estimation of the force.

In accordance with one of some embodiments, there is provided a notification method comprising:
acquiring color information of a target tissue from a captured image output from an imager capturing an image of a tissue, the target tissue being a portion of the tissue that covers at least a portion of an end effector of a treatment tool that has slid into the tissue;
estimating a force applied to the target tissue by the end effector from the color information of the target tissue; and
notifying an operator of a result of the estimation of the force.

### BRIEF DESCRIPTION OF THE DRAWINGS

FIG. 1 is a view for describing an outline of a support method in the present embodiment.

FIG. 2 shows a configuration example of a medical system in a first embodiment.

FIG. 3 shows a first configuration example of a controller in the first embodiment.

FIG. 4 is a flowchart of processing in the first configuration example.

FIG. 5 is a diagram for describing details of processing executed by a first measurement section.

FIG. 6 shows a first detailed example regarding recognition of a treatment tool and setting of a region of interest.

FIG. 7 shows a second detailed example regarding recognition of a treatment tool and setting of a region of interest.

FIG. 8 shows a third detailed example regarding recognition of a treatment tool and setting of a region of interest.

FIG. 9 shows another example of step S2b in a case where a hook is not seen in an endoscope image.

FIG. 10 shows another example of step S2b in a case where the hook is not seen in an endoscope image.

FIG. 11 shows a fourth detailed example regarding recognition of a treatment tool and setting of a region of interest.

FIG. 12 shows a fifth detailed example regarding recognition of a treatment tool and setting of a region of interest.

FIG. 13 shows a third detailed example regarding recognition of a treatment tool and setting of a region of interest.

FIG. 14 is a diagram for describing details of processing executed by a second measurement section, a determination section, and a presentation section.

FIG. 15 shows a first detailed example of tension determination.

FIG. 16 shows a second detailed example of tension determination.

FIG. 17 shows a second configuration example of a controller in the first embodiment.

FIG. 18 is a flowchart of processing in the second configuration example.

FIG. 19 is a diagram for describing details of processing executed by a tissue determination section and a switching section in the second configuration example.

FIG. 20 is a flowchart of processing in the third configuration example.

FIG. 21 is a diagram for describing details of processing executed by a tissue determination section and a switching section in the third configuration example.

FIG. 22 shows a configuration example of a medical system in a second embodiment.

FIG. 23 shows a first configuration example of a controller in the second embodiment.

FIG. 24 is a flowchart of processing in a first configuration example of the second embodiment.

FIG. 25 shows a second configuration example of a controller in the second embodiment.

FIG. 26 shows a third configuration example of a controller in the second embodiment.

FIG. 27 is a first flowchart of processing in a third configuration example of the second embodiment.

FIG. 28 is a first flowchart of processing in a third configuration example of the second embodiment.

FIG. 29 shows a fourth configuration example of a controller in the second embodiment.

FIG. 30 is a flowchart of processing in a fourth configuration example of the second embodiment.

FIG. 31 is a flowchart of processing in a third embodiment.

FIG. 32 is a configuration example of a medical system and a controller in a fourth embodiment.

FIG. 33 is a flowchart of a first detailed example of the fourth embodiment.

FIG. 34 is a flowchart of a first detailed example of the fourth embodiment.

FIG. 35 shows an example of a presentation method.

FIG. 36 shows an example of a presentation method.

FIG. 37 shows an example of a presentation method.

FIG. 38 is a flowchart of a second detailed example of the fourth embodiment.

FIG. 39 shows a detailed example of steps S52 and S53.

FIG. 40 shows an example of allocating a function of giving approval or rejection and a function of performing energy output to one button.

FIG. 41 is a flowchart of a third detailed example of the fourth embodiment.

FIG. 42 shows an example of providing delay time starting, not from pressing of a button, but from presentation of information to the start of output.

FIG. 43 is a flowchart of a fourth detailed example of the fourth embodiment.

### DETAILED DESCRIPTION

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

### 1. Method

In a manipulation using a hook-knife type monopolar device, a skill is required to obtain a sense of an appropriate tension, and there is a case where an unskilled physician cannot perform surgery efficiently. Hence, appropriate support is desired to be given to an operator. Note that Japanese Unexamined Patent Application Publication No. 2014-213094 relates to support regarding lesion evaluation, but not relates to support regarding a manipulation using a hook-knife type monopolar device. While the description herein is given taking the hook-knife type monopolar device as an example, a similar issue occurs in a situation where energy is applied while a tissue is pulled for a treatment on the tissue.

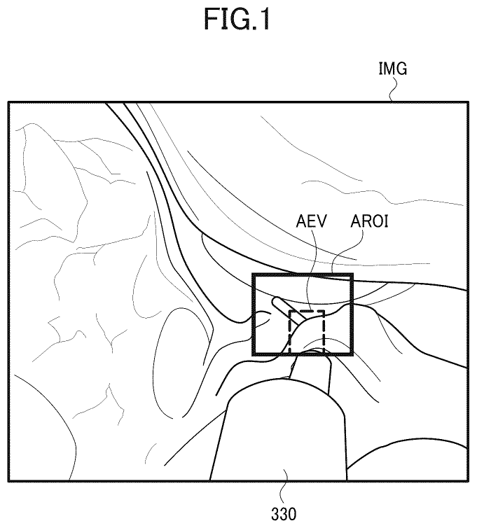

FIG. 1 is a view for describing an outline of a support method in the present embodiment.

When a tissue is incised with use of the hook-knife type monopolar device, there is a possibility that an unskilled physician does not know an appropriate tension in comparison with a skilled physician. Thus, a system of making notification about an appropriate tension is desirable for an operator.

As shown in FIG. 1, the medical system in the present embodiment uses treatment tool recognition with artificial intelligence (AI) or the like to detect a region of interest AROI from an endoscope image IMG. The region of interest AROI is set at the periphery of a distal end of a hook knife 330. In a region AEV corresponding to the distal end of the hook knife 330, the distal end of the hook knife 330 is transparently seen through the tissue. In addition, the tissue is pulled with the distal end of the hook knife 330, whereby a tension occurs in the tissue. The medical system obtains transmittance of the distal end of the hook knife 330 transparently seen through the tissue from an image of the region of interest AROI, and estimates a tensile force in the tissue from the transmittance. The medical system then determines whether or not a tension applied to the tissue at the periphery of the distal end of the hook knife 330 is appropriate based on the estimated tensile force, and presents a result through monitor display or the like to the operator.

In accordance with the present embodiment, the tension applied to the tissue by the hook knife 330 is estimated from an endoscope image, and support information is presented to the operator based on the estimated tension. With this configuration, it is possible to implement support for implementing a safe and efficient tension regardless of a level of skill of an operating surgeon when lifting the tissue with use of the hook knife 330 to perform coagulation incision. That is, tension determination appropriate for a treatment, which is implicit knowledge of the skilled physician, is made on the system side, whereby the unskilled physician can use the hook-knife type monopolar device safely and efficiently.

In addition, since the region of interest AROI is set at the periphery of the distal end of the hook knife 330, an image used for the tension determination is limited to only an image of the periphery of the distal end of the hook knife 330. This enables appropriate tension evaluation from the endoscope image. That is, the region of interest AROI is set, not at a freely-selected location on the tissue, but at the distal end of the treatment tool where recognition is relatively easy, and only the tissue within the region of interest AROI serves as an evaluation target, whereby only a location on which the operator wants to perform a treatment can be stably evaluated.

Additionally, with use of a color of a distal end portion of the hook knife 330 that is transparently seen on the back side of the tissue pulled by the hook knife 330 as a criterion, it becomes possible to more easily determine whether or not a tension applied to the tissue is appropriate.

### 2. First Embodiment

### 2.1. Medical System

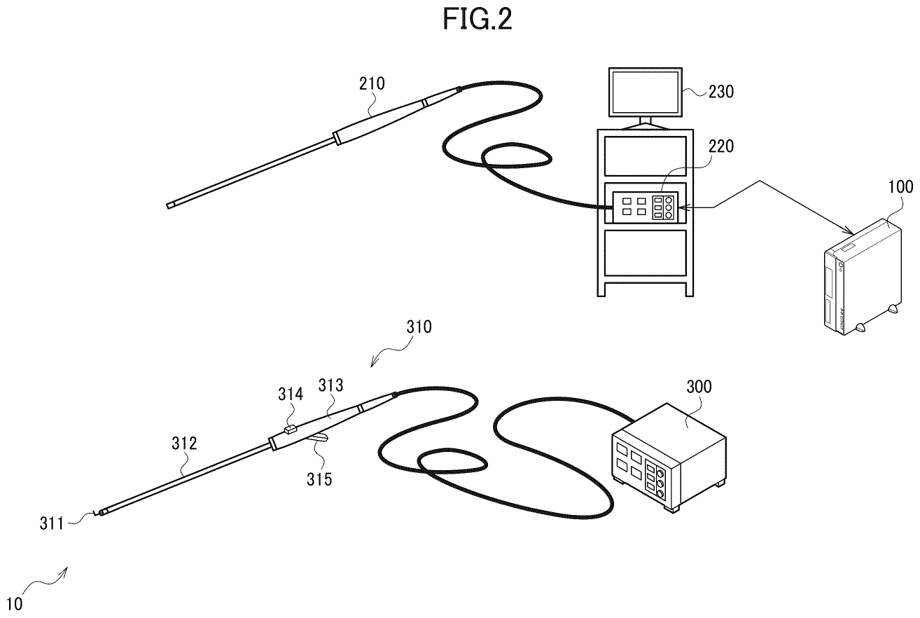

FIG. 2 shows a configuration example of the medical system in a first embodiment. A medical system 10 includes a controller 100, an endoscope 210, a main body device 220 of the endoscope, a monitor 230, a generator 300, and a treatment tool 310. A system using a rigid scope for surgery is described herein, but the support method of the present embodiment can be applied to a system using a flexible scope for the digestive tract. In this case, a shaft of the treatment tool 310 includes a long-axis flexible member. The shaft passes through a treatment tool channel of the flexible scope, and the distal end portion of the treatment tool 310 protrudes from the distal end of the flexible scope.

The endoscope 210 is a rigid scope that is inserted into a body cavity and that captures an image of the inside of the body cavity. The endoscope 210 includes, for example, an insertion portion to be inserted into the body cavity, an operation portion to be connected to the base end of the insertion portion, a universal cord connected to the base end of the operation portion, and a connector portion to be connected to the base end of the universal cord. An imaging device for capturing the image of the inside of the body cavity and an illumination optical system for illuminating the inside of the body cavity are arranged in a distal end of the insertion portion. The imaging device includes an objective optical system and an imager that captures an object image formed by the objective optical system. The connector portion detachably connects the transmission cable to the main body device 220. The image captured by the endoscope 210 is hereinafter referred to as a captured image or an endoscope image.

The main body device 220 includes a processing device that controls the endoscope and that performs image processing on the endoscope image and processing of displaying the endoscope image, and a light source device that generates and controls illumination light. The processing device includes a processor such as a central processing unit (CPU), and performs image processing on an image signal transmitted from the endoscope 210 to generate the endoscope image and then outputs the endoscope image to the monitor 230 and the controller 100. The illumination light emitted from the light source device is guided by a light guide to the illumination optical system of the endoscope 210 and is emitted from the illumination optical system into the inside of the body cavity. The monitor 230 is, for example, a liquid crystal display, an organic electroluminescence (EL) display, or the like.

The treatment tool 310 is a device that outputs energy with high-frequency power, ultrasound waves, or the like from its distal end portion to perform a treatment, such as coagulation, sealing, hemostasis, incision, division, and dissection, on a tissue in contact with its distal end portion. The treatment tool 310 is also referred to as an energy device. The description herein is given of an example in which the treatment tool 310 is the hook-knife type monopolar device. The monopolar device mentioned herein is a device that carries high-frequency power between an electrode at the distal end of the device and an electrode outside the body. However, the treatment tool 310 is only required to be an energy device that applies energy while applying a tensile force to the tissue by causing an end effector to slide into the tissue to treat the tissue.

The treatment tool 310 includes a shaft 312, an end effector 311 connected to the distal end side of the shaft 312, and a handle portion 313 connected to the base end side of the shaft 312. The shaft 312 has a thin, long, cylindrical shape, and made of a hard member. The end effector 311 has a hook shape so as to cause the end effector 311 to slide into the tissue and pull the tissue. The end effector 311 corresponds to the electrode of the monopolar device. The end effector 311 applies energy to the tissue in a state of pulling the tissue, and thereby performs incision, coagulation, or the like on the tissue. The handle portion 313 is an operation portion to be gripped by the operator to operate the treatment tool 310. The handle portion 313 is, for example, of a straight type, that is, has a substantially cylindrical shape that is coaxial with the shaft 312, but is not limited thereto and may have various kinds of shapes. For example, the handle portion 313 may be of a gun type in which a handle protrudes in a direction crossing the shaft 312, or of another shape. The handle portion 313 is provided with a button 314 used by the operator to give an instruction for energy output or the like. FIG. 2 shows an example in which one button is provided, but a plurality of buttons may be provided. In a case where an external switch such as a foot switch is used, the button may be omitted from the handle portion 313. The handle portion 313 may be provided with a port 315 for air supply and air intake. In this case, a channel is provided so as to extend from the port 315 to the distal end of the shaft 312 while penetrating the handle portion 313 and the shaft 312.

The generator 300 supplies energy to the treatment tool 310, controls energy supply, and acquires impedance information. That is, the generator 300 outputs high-frequency power, and the treatment tool 310 outputs the high-frequency power from the end effector 311. An output sequence in accordance with a type of a treatment such as incision and coagulation or an output level in each output sequence is set in the generator 300. These settings can be changed, for example, via an operation portion arranged in the generator 300. The generator 300 outputs high-frequency power in accordance with the set output sequence or the set output level. The impedance information can be obtained from a relationship between voltage and current when the treatment tool 310 outputs high-frequency power to the tissue.

The controller 100 generates support information regarding a tension from the endoscope image and displays the support information on the monitor as described with reference to FIG. 1. The support information may be displayed on the monitor 230 of the endoscope, or may be displayed on a monitor that is not shown and that is separately provided to display various kinds of support information. The controller 100 is, for example, an information processing device such as a personal computer. Note that FIG. 2 shows the example in which the main body device 220 of the endoscope and the controller 100 are different devices, but a configuration may be adopted in which functions of the controller 100 are built into the main body device 220. Note that the following description will be given of an example in which the support information is displayed on the monitor 230 of the endoscope, and the main body device 220 and the controller 100 are different devices.

### 2.2. First Configuration Example

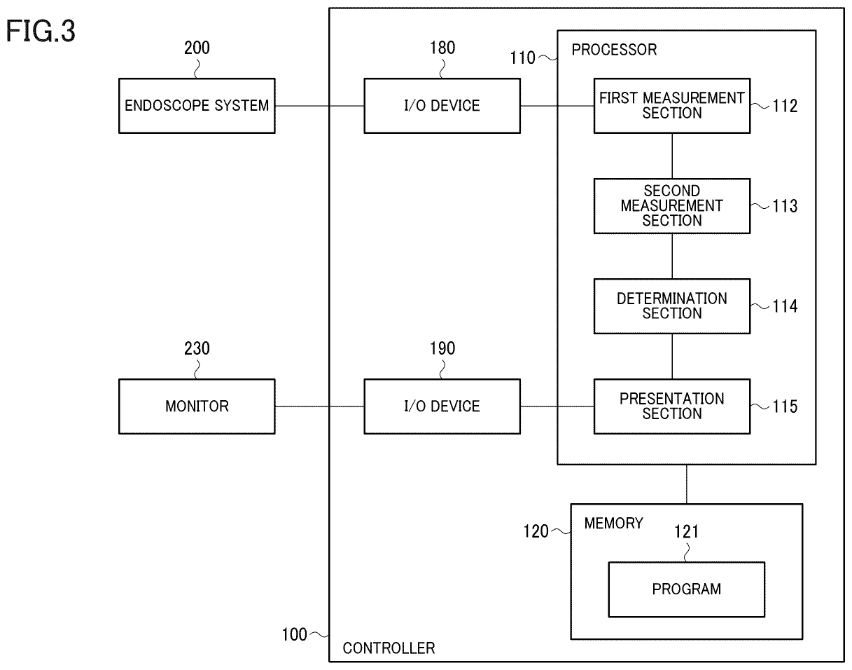

FIG. 3 shows a first configuration example of the controller in the first embodiment. An endoscope system 200 corresponds to the endoscope 210 and the main body device 220 shown in FIG. 2. The controller 100 includes a processor 110, a memory 120, an input/output (I/O) device 180, and an I/O device 190. Note that illustration of the generator 300 and the treatment tool 310 is omitted here.

The I/O device 180 receives an endoscope image from the endoscope system 200. The I/O device 180 is a cable connector to be connected to the main body device 220, or a communication circuit that performs processing of communicating with the main body device 220.

The I/O device 190 outputs image data output from the processor 110 to the monitor 230. The monitor 230 performs image display based on image data received from the I/O device 190. The image data includes support information regarding a tension applied to the tissue by the hook-knife type monopolar device. In addition, the image data may further include an endoscope image. The I/O device 190 is a cable connector to be connected to the monitor 230, or a communication circuit that performs processing of communicating with the monitor 230.

The processor 110 includes hardware. The processor 110 is, for example, a central processing unit (CPU), a graphics processing unit (GPU), a microcomputer, a digital signal processor (DSP), or the like. Alternatively, the processor 110 may be an application-specific integrated circuit (ASIC), a field programmable gate array (FPGA), or the like. The processor 110 may include one or more of the CPU, the GPU, the microcomputer, the DSP, the ASIC, the FPGA, and the like. The memory 120 is, for example, a semiconductor memory, which is a volatile memory or a non-volatile memory. Alternatively, the memory 120 may be a magnetic storage device such as a hard disk device, or may be an optical storage device such as an optical disk device.

The memory 120 stores a program 121 in which various kinds of processing contents are described. The processor 110 executes the program 121 to execute various kinds of processing. The program 121 may include a trained model obtained by machine learning. The trained model may include, for example, a program in which algorithms of AI are described, data used in the program, and the like. For example, the trained model may include a neural network such as a convolutional neural network (CNN). In this case, the trained model includes a program in which algorithms of the neural network are described, a weight parameter assigned between nodes of the neural network, and the like. Note that a non-transitory information storage medium, which is a computer-readable storage medium, may store the program 121. The information storage medium is, for example, an optical disk, a memory card, a hard disk drive, a semiconductor memory, or the like. The semiconductor memory is, for example, a read-only memory (ROM) or a non-volatile memory. The controller 100 loads the program 121 stored in the information storage medium in the memory 120, and performs various kinds of processing based on the program 121.

In the first configuration example, the memory 120 stores the program 121 in which processing contents of a first measurement section 112, a second measurement section 113, a determination section 114, and a presentation section 115 are described. The processor 110 executes the program 121 to execute processing of each of the first measurement section 112, the second measurement section 113, the determination section 114, and the presentation section 115. For example, the program 121 includes program modules in which processing of each section is described, and the processor 110 executes the program modules to execute the processing of each section.

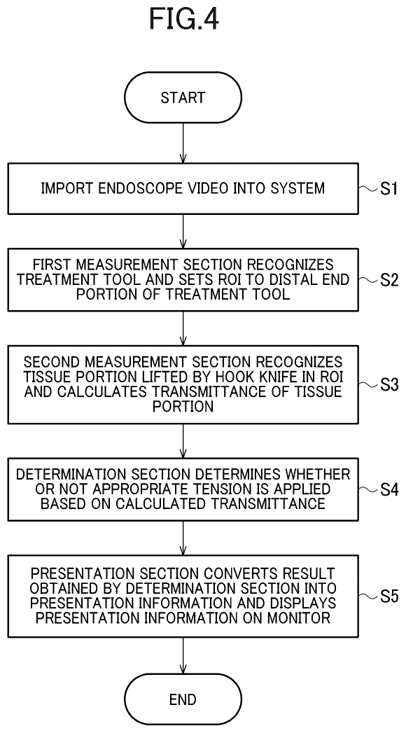

FIG. 4 is a flowchart of processing in the first configuration example. Note that “ROI” in FIG. 4 represents a region of interest.

In step S1, the I/O device 180 imports an endoscope video captured by the endoscope system 200. In addition, the first measurement section 112 acquires an endoscope image imported by the I/O device 180. In step S2, the first measurement section recognizes a treatment tool seen in the endoscope image, and sets a region of interest in the vicinity of a distal end portion of the recognized treatment tool.

In step S3, the second measurement section 113 uses an image of the region of interest to calculate transmittance of a hook in a tissue portion lifted by the hook-knife type monopolar device based on the image of the region of interest. For example, the second measurement section 113 recognizes the tissue portion lifted by the hook-knife type monopolar device within the region of interest, and calculates the transmittance of the hook in the recognized tissue portion.

In step S4, the determination section 114 determines whether or not an appropriate tension is applied to the tissue based on the calculated transmittance.

In step S5, the presentation section 115 converts a result of the determination made by the determination section 114 into presentation information, and displays the presentation information on the monitor 230. The presentation information is, for example, image information including characters, figures, colors, icons, and the like.

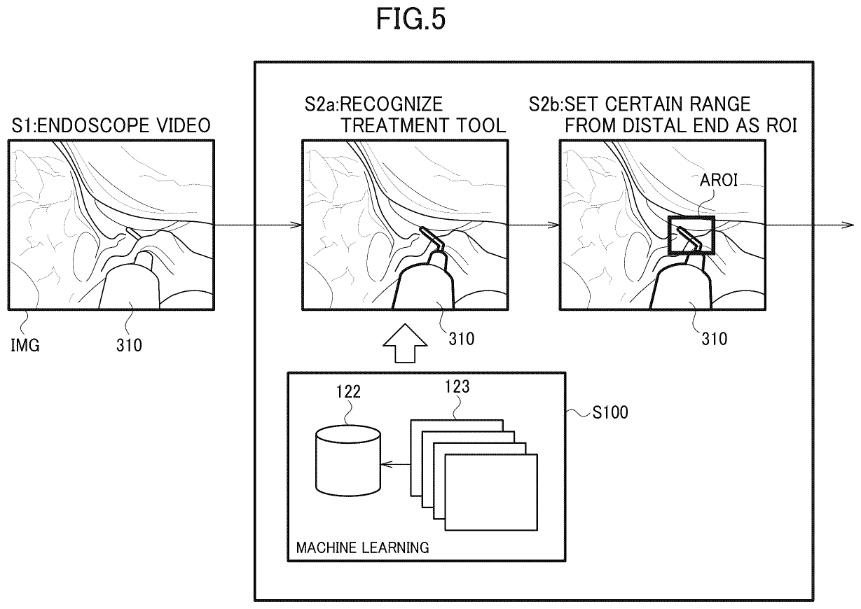

Details of processing performed by each section will be described below. FIG. 5 is a diagram for describing details of processing executed by the first measurement section.

In step S1, the first measurement section 112 acquires the endoscope image IMG. The endoscope image IMG is a frame image of moving images captured by the endoscope 210. Basically, processing in steps S1 to S5 is executed on moving images in real time. Note that, for example, the processing in steps S1 to S5 may be executed on recorded moving images.

In step S100, a trained model 122 is generated by machine learning. This step S100 is executed in a training phase, and may not be included in step S2 that is in an inference phase and shown in FIG. 4. Training data 123 includes a plurality of endoscope images and an annotation added to each endoscope image. Object detection by segmentation is mainly assumed, and the annotation is data that indicates a region of the treatment tool in an endoscope image. Specifically, the annotation is added to each of a region of the shaft and a region of the hook. The machine learning is executed by a training device such as an information processing device. A processor of the training device inputs an endoscope image of the training data 123 to a training model, which detects the region of the treatment tool from the endoscope image, and updates a weight parameter of the training model based on an error between a result of the detection and the annotation of the training data 123. The training device repeats this updating processing on multitudes of endoscope images to generate the trained model 122.

In step S2a, the first measurement section 112 inputs the endoscope image IMG to the trained model 122, and the trained model 122 detects a region of the treatment tool 310 from the endoscope image IMG. In step S2b, the first measurement section 112 sets the region of interest AROI on the endoscope image IMG based on the detected region of the treatment tool 310. The region of interest AROI is a predetermined range with the distal end of the treatment tool 310 serving as a criterion. The shape of the region of interest AROI may be, as described later, a rectangle, a circle, a shape of the hook itself, or the like.

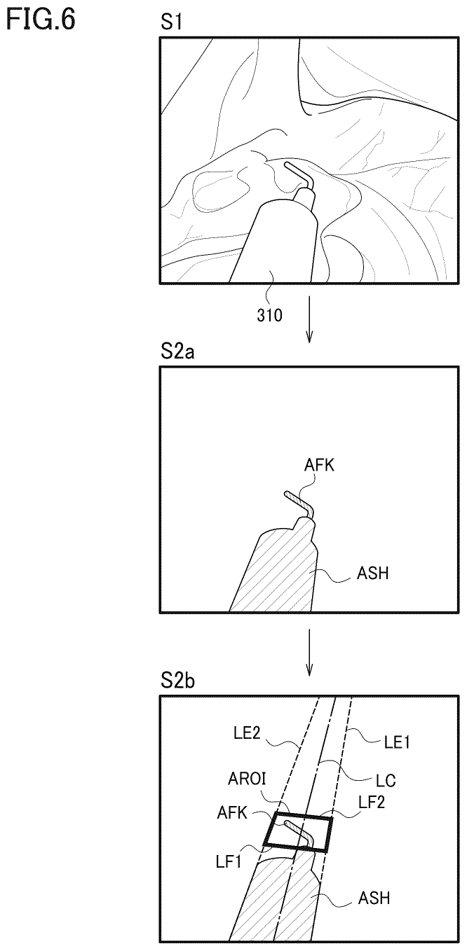

FIG. 6 shows a first detailed example regarding recognition of the treatment tool and setting of the region of interest. Note that in the machine learning in step S100, performed is training for segmentation on an endoscope image in which the whole of the hook is seen, an endoscope image in which the hook is partially hidden under the tissue and a portion of the hook is seen, and an endoscope image in which the whole of the hook is hidden under the tissue and the hook is not seen. In the detection performed by the trained model, a result of segmentation depending on a state of the hook seen in the endoscope image is output.

In step S1 in FIG. 6, assumed is a case where the hook has not slid into the tissue, and the whole of the hook is seen in the endoscope image.

In step S2a, the first measurement section 112 performs segmentation using machine learning to detect a region ASH of the shaft of the treatment tool and a region AFK of the hook of the treatment tool from the endoscope image.

In step S2b, the first measurement section 112 calculates a principal component long-axis vector LC, a first edge extension line LE1, and a second edge extension line LE2 from the region ASH of the shaft. The principal component long-axis vector LC is a center line in a long-axis direction of the region ASH of the shaft. The first edge extension line LE1 and the second edge extension line LE2 are lines extended from the respective edges of the region ASH of the shaft in the long-axis direction of the region ASH of the shaft. In addition, the first measurement section 112 calculates a hook base end line LF1 and a hook distal end line LF2 from the region AFK of the hook. The hook base end line LF1 is a line passing through the base end of the region AFK of the hook and perpendicular to the principal component long-axis vector LC. The hook distal end line LF2 is a line passing through the distal end of the region AFK of the hook and perpendicular to the principal component long-axis vector LC. The first measurement section 112 sets a rectangular region surrounded by the first edge extension line LE1, the second edge extension line LE2, the hook base end line LF1, and the hook distal end line LF2 as the region of interest AROI.

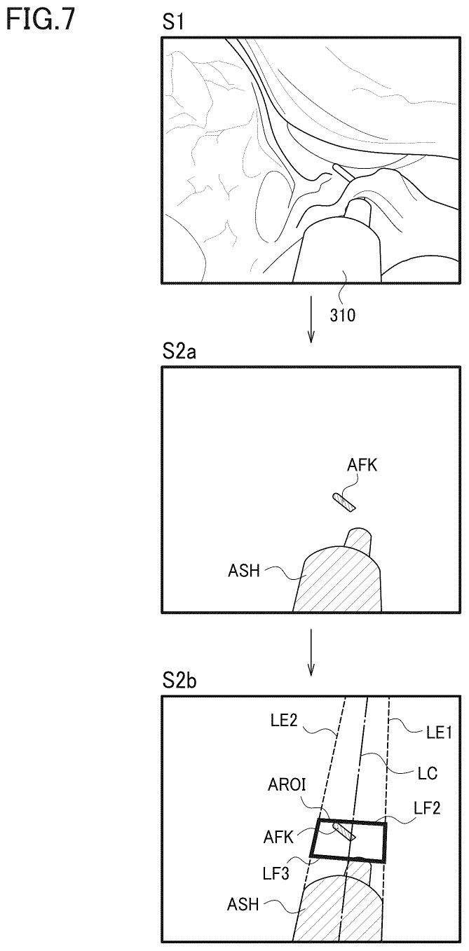

FIG. 7 shows a second detailed example regarding recognition of the treatment tool and setting of the region of interest. In step S1 in FIG. 7, assumed is a case where a portion of the hook on the base end side has slid into the tissue, and a portion of the hook on the distal end side is seen in the endoscope image.

In step S2a, the first measurement section 112 performs segmentation using machine learning to detect the region ASH of the shaft of the treatment tool and the region AFK of the hook of the treatment tool from the endoscope image. The region AFK of the hook is a region in which the portion of the hook on the distal end side seen in the endoscope image has been subjected to the segmentation. That is, in the segmentation, a region corresponding to the hook on the base end side is not detected.

In step S2b, the first measurement section 112 calculates, in substitution for the hook base end line LF1, a shaft distal end line LF3 from the region ASH of the shaft. The shaft distal end line LF3 is a line passing through the distal end of the region ASH of the shaft and perpendicular to the principal component long-axis vector LC. The other lines are similar to those in FIG. 6. The first measurement section 112 sets a rectangular region surrounded by the first edge extension line LE1, the second edge extension line LE2, the shaft distal end line LF3, and the hook distal end line LF2 as the region of interest AROI.

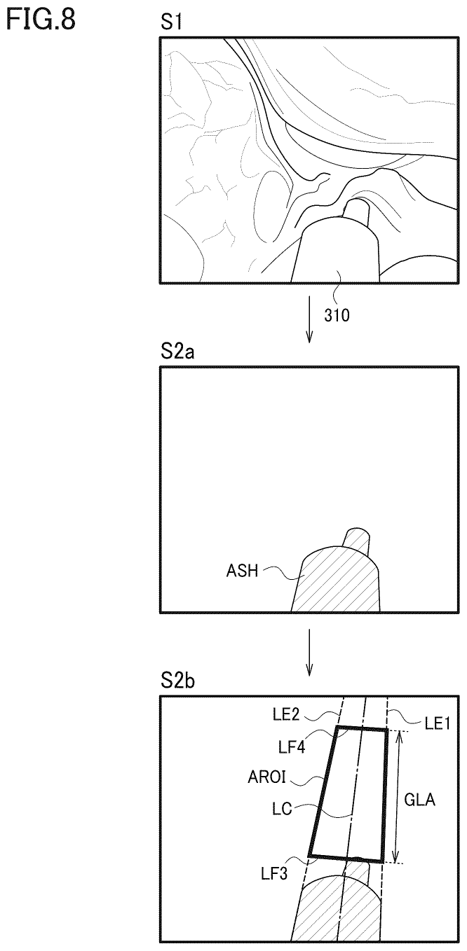

FIG. 8 shows a third detailed example regarding recognition of the treatment tool and setting of the region of interest. In step S1 in FIG. 8, assumed is a case where the whole of the hook has slid into the tissue, and the hook is not seen in the endoscope image.

In step S2a, the first measurement section 112 performs segmentation using machine learning to detect the region ASH of the shaft of the treatment tool from the endoscope image. Since the hook is not seen in the endoscope image, the region AFK of the hook is not detected.

In step S2b, the first measurement section 112 calculates the principal component long-axis vector LC, the first edge extension line LE1, the second edge extension line LE2, and the shaft distal end line LF3 from the region ASH of the shaft. The first measurement section 112 sets a line LF4 away from the shaft distal end line LF3 by a fixed length GLA. The line LF4 is a line set closer to the distal end than the shaft distal end line LF3 is to the distal end, and perpendicular to the principal component long-axis vector LC. The fixed length GLA is, for example, a length represented by the number of pixels or the like on the image. The first measurement section 112 sets a rectangular region surrounded by the first edge extension line LE1, the second edge extension line LE2, the shaft distal end line LF3, and the line LF4 as the region of interest AROI.

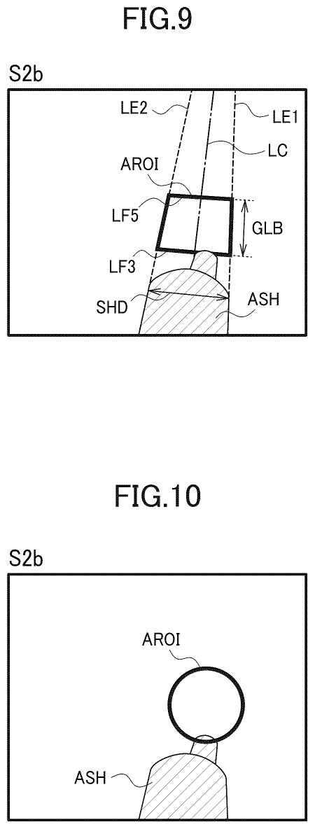

FIGS. 9 and 10 each show another example of step S2b in a case where the hook is not seen in the endoscope image.

In the example in FIG. 9, the first measurement section 112 calculates the principal component long-axis vector LC, the first edge extension line LE1, the second edge extension line LE2, and the shaft distal end line LF3 from the region ASH of the shaft. The first measurement section 112 calculates a shaft diameter SHD on the image from the region ASH of the shaft, multiplies the shaft diameter SHD by a predetermined magnification ratio to calculate a length GLB, and sets a line LF5 away from the shaft distal end line LF3 by the length GLB. The line LF5 is a line set closer to the distal end than the shaft distal end line LF3 is to the distal end, and perpendicular to the principal component long-axis vector LC. The first measurement section 112 sets a rectangular region surrounded by the first edge extension line LE1, the second edge extension line LE2, the shaft distal end line LF3, and the line LF5 as the region of interest AROI.

In the example in FIG. 10, the first measurement section 112 sets, as the region of interest AROI, a predetermined region with the distal end of the region ASH of the shaft serving as a criterion. The predetermined region is, for example, a region centering on a point away from the distal end of the region ASH of the shaft by a predetermined distance. The shape of the predetermined region may be a circle as shown in FIG. 10, or may be a rectangle.

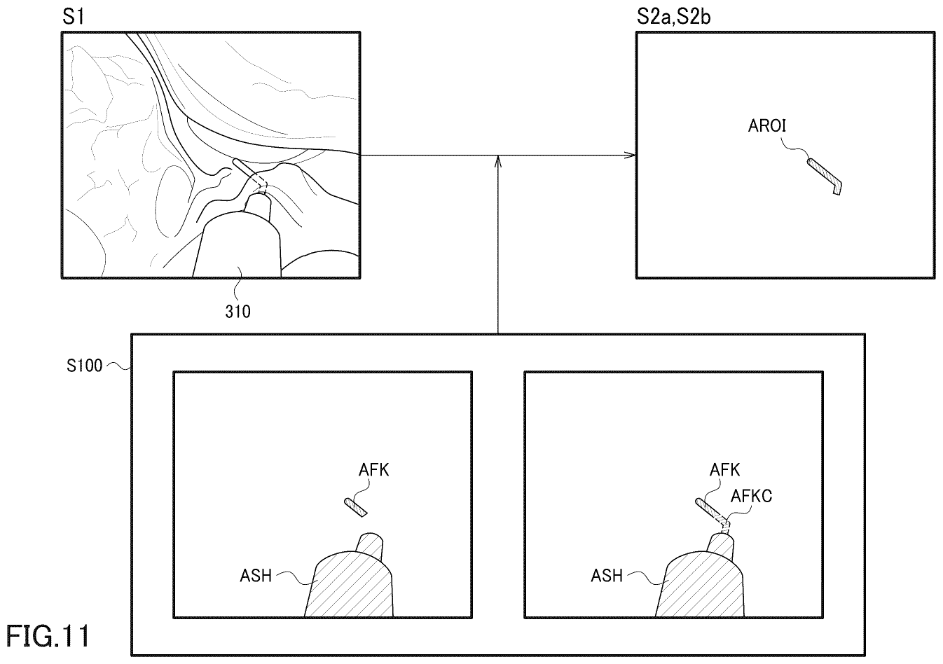

FIG. 11 shows a fourth detailed example regarding recognition of the treatment tool and setting of the region of interest. In this example, the region of the hook including the portion hidden by the tissue is detected, and the region of the hook itself is set as the region of interest AROI.

In step S100 in the training phase, created are a piece of data subjected to annotation of the region AFK corresponding to the portion of the hook seen in the endoscope image, and a piece of data subjected to annotation of the whole of the hook by interpolation of the region AFK with a region AFKC corresponding to the portion of the hook not seen in the endoscope image (that is, AFK+AFKC). The training device trains the training model with a pair of these pieces of annotation data and the endoscope image.

In step S2a in the inference phase, the first measurement section 112 performs segmentation using the trained model to detect the region of the whole of the hook including the portion of the hook not seen in the endoscope image from the endoscope image. In step S2b, the first measurement section 112 sets the region of interest AROI to the detected region of the whole of the hook.

Note that the description herein has been given of the example in which the portion of the hook on the distal end side is seen in the endoscope image. In a case where the whole of the hook is seen in the endoscope image, the region of the whole of the hook seen in the endoscope image may be detected by segmentation, and the detected region may be set as the region of interest AROI. In a case where the hook is not seen in the endoscope image, training may be performed using data subjected to annotation of a hook portion not seen in the endoscope image, the region of the whole of the hook not seen in the endoscope image may be detected by the trained model, and the detected region may be set as the region of interest AROI.

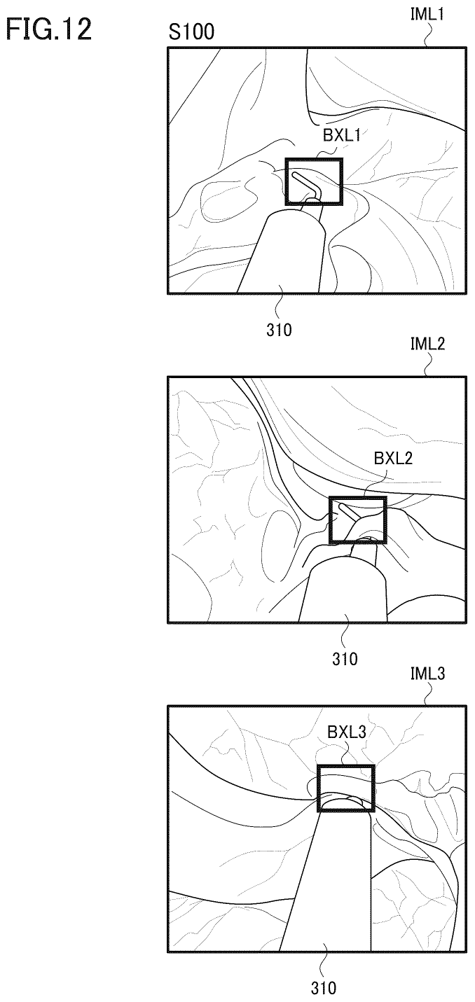

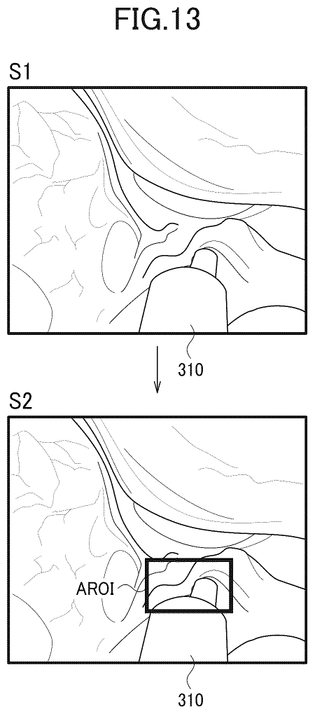

FIGS. 12 and 13 each show a fifth detailed example regarding recognition of the treatment tool and setting of the region of interest. In this example, the region of interest is set directly from the endoscope image not by using the recognition of the treatment tool by segmentation, but by using detection or the like. While the detection method using the detection will be described below, an algorithm of machine learning is only required to an algorithm that can output a result of inference of the region of interest with respect to image input.

As shown in FIG. 12, in step S100 in the training phase, created is data subjected to annotation of boundary boxes BXL1 to BXL3 surrounding the respective hooks seen in endoscope images IML1 to IML3, respectively. The endoscope image IML1 is an example in which the whole of the hook is seen in the endoscope image. The endoscope image IML2 is an example in which a portion of the hook is hidden under the tissue. The boundary box BXL2 is set so as to include the whole of the hook including the portion hidden under the tissue. The endoscope image IML3 is an example in which the whole of the hook is hidden under the tissue. The boundary box BXL3 is set so as to include the whole of the hook hidden under the tissue. The training device trains the training model with these endoscope images and annotation data serving as training data.

As shown in FIG. 13, in step S2 in the inference phase, the first measurement section 112 performs the detection using the trained model to detect a boundary box corresponding to the hook portion. The first measurement section 112 sets the inside of the detected boundary box as the region of interest AROI.

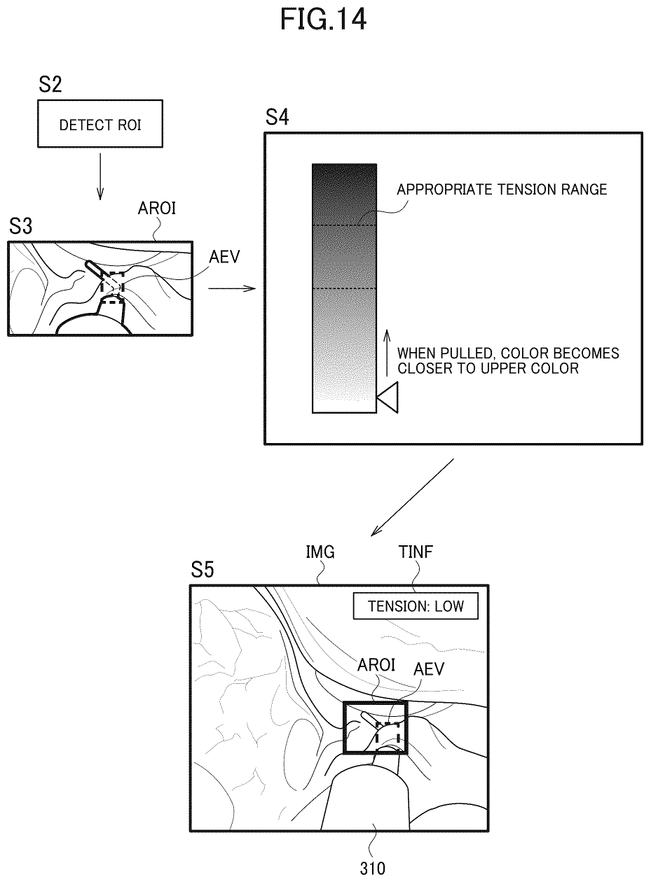

FIG. 14 is a diagram for describing details of processing executed by each of the second measurement section, the determination section, and the presentation section.

In step S3, the second measurement section 113 acquires color information of the region AEV corresponding to a portion into which the hook has slid from the image of the region of interest AROI. For example, the second measurement section 113 extracts a region in an intermediate color between a color of the hook and a color of the tissue in the region of interest AROI, determines the region AEV, and acquires color information of the image of the region AEV. Alternatively, in a case where the region of the hook is extracted directly as the region of interest AROI as shown in FIG. 11, the second measurement section 113 may acquire the color information of the region of interest AROI as the color information of the portion into which the hook has slid without setting the region AEV. The color information is, for example, a red, green, and blue (RGB) value, a YCrCb value, a hue, saturation, and brightness (HSV) value, or the like. In addition, the color information is an average value, a representative value, or the like in the region AEV. The second measurement section 113 calculates transmittance of the hook with the color of the tissue and the color of the hook serving as a criterion based on the color information of the region AEV. That is, the second measurement section 113 evaluates how much the color of the hook is mixed with the color of the tissue, and calculates the transmittance of the hook that is transparent through the tissue. The color of the tissue serving as the criterion is acquired from, for example, the color information of the tissue at the periphery of the region of interest AROI, and the color of the hook is acquired from, for example, the color information of the hook seen in the image. Alternatively, the color of the tissue serving as the criterion may be a predetermined color that is preliminarily set, and the color of the hook may be a predetermined color that is preliminarily set. Information about these predetermined colors may be stored in the memory 120, a database, or the like. The second measurement section 113, for example, expresses the color information of the region AEV with α blending of the color of the tissue and the color of the tissue, and calculates the transmittance with a blending ratio α.

In step S4, the determination section 114 determines whether or not a tension in the tissue pulled by the hook-knife type monopolar device is within an appropriate tension range based on the transmittance calculated from the image. A lower end of gradation shown in S4 indicates the color of the tissue and an upper end thereof indicates the color of the hook. Since the color becomes lighter when a tension is applied to the tissue, the transmittance of the hook becomes higher, and the color information of the region AEV becomes closer to the color of the hook at the upper end. Based on the above, the tension can be evaluated from the transmittance.

In step S5, the presentation section 115 displays a result of evaluation of the tension on the monitor. For example, the presentation section 115 generates image data in which presentation information TINF is added to the endoscope image IMG, and outputs the image data on the monitor. The presentation section 115, for example, displays “TENSION: LOW” when the transmittance is lower than a lower limit of the appropriate range, displays “TENSION: APPROPRIATE” when the transmittance is within the appropriate range, and displays “TENSION: HIGH” when the transmittance is higher than an upper limit of the appropriate range. FIG. 14 shows the example in which the presentation information TINF is character information, but may be a color, an icon, or the like. Alternatively, the presentation section 115 may present a result of evaluation of the tension to the operator, not through monitor display, but through sound, vibration, or the like.

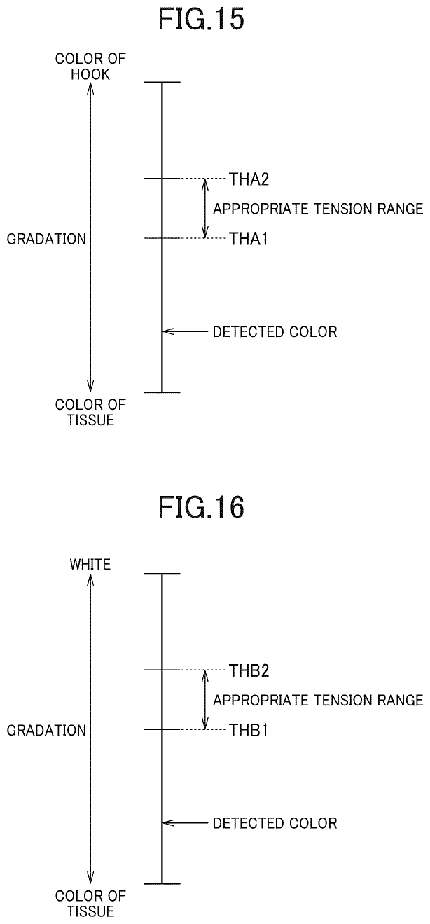

FIG. 15 shows a first detailed example of tension determination. As described above, the second measurement section 113 calculates the transmittance of the hook transparently seen through the tissue from the color of the tissue, the color of the hook, and the detected color. Assume that a lower limit value of the transmittance indicating the appropriate tension range is THA1, and an upper limit value thereof is THA2. The determination section 114 makes a comparison between the transmittance and the lower limit value THA1 and a comparison between the transmittance and the upper limit value THA2. When a relation of THA1≤the transmittance≤THA2 holds, the determination section 114 determines that the tension is appropriate. When a relation of the transmittance <THA1 or a relation of THA2<the transmittance holds, the determination section 114 determines that the tension is inappropriate. More specifically, when the relation of the transmittance <THA1 holds, the determination section 114 determines that the tension is low. When the relation of THA2<the transmittance holds, the determination section 114 determines that the tension is high.

FIG. 16 shows a second detailed example of tension determination. In this example, whitening of the tissue due to pulling is evaluated, whereby a tension is evaluated. The second measurement section 113 calculates a degree of whitening of the tissue with the color of the tissue and white serving as a criterion based on the color information of the region AEV corresponding to the portion into which the hook has slid. The degree of whitening indicates how much white is mixed with the color of the tissue. The second measurement section 113, for example, expresses the color information of the region AEV with α blending of the color of the tissue and white, and calculates the degree of whitening with a blending ratio α. Assume that a lower limit value of the degree of whitening indicating the appropriate tension range is THB1, and an upper limit value thereof is THB2. The determination section 114 makes a comparison between the degree of whitening and the lower limit value THB1 and a comparison between the degree of whitening and the upper limit value THB2. When a relation of THB1≤the degree of whitening≤THB2 holds, the determination section 114 determines that the tension is appropriate. When a relation of the degree of whitening <THB1 or a relation of THB2<the degree of whitening holds, the determination section 114 determines that the tension is inappropriate. More specifically, when the relation of the degree of whitening <THB1 holds, the determination section 114 determines that the tension is low. When the relation of THB2<the degree of whitening holds, the determination section 114 determines that the tension is high.

Note that the tension is evaluated from an index value itself such as the transmittance and a color in the above description, but may be evaluated from an amount of change of the index value as described in the following examples.
First example: When the tissue is pulled, there is a possibility that the color of the tissue is changed not only to white, but also to various tinges. The second measurement section 113 may evaluate the change of the color of the tissue before and after the pulling regardless of which color the tissue has after the pulling to evaluate the tension. For example, the second measurement section 113 may evaluate a difference between the color of the tissue before the pulling and the color of the tissue after the pulling.
Second example: In the evaluation of the tension using the transmittance, the transmittance before the pulling is not necessarily zero. For example, in a tissue having high transmittance such as a thin membrane, there is a possibility that the transmittance is about 50% at a time point when the hook knife is inserted under the thin membrane. In contrast, in a tissue having low transmittance, there is a possibility that the transmittance is close to zero at a time point when the hook knife is inserted under the thin membrane. The second measurement section 113 may evaluate, not the transmittance itself, but a change of the transmittance before and after the pulling to evaluate the tension. For example, the second measurement section 113 may evaluate a difference between the transmittance before the pulling and the transmittance after the pulling. In the above-mentioned example of the thin membrane, for example, the second measurement section 113 may determine that the tension becomes the appropriate tension when the transmittance of 50% before the pulling becomes the transmittance of 60% after the pulling.
Third example: The second measurement section 113 may evaluate opacity=100%-transparency regardless of the change of the transmittance, and may use, for example, such an amount of change as that opacity after the pulling is 0.8 times opacity before the pulling as the index.

As another example of the tension determination, an example of using an optical flow is now described. The second measurement section 113 detects a difference between an optical flow of the tissue and an optical flow of the treatment tool from the image of the region of interest AROI. The determination section 114 determines whether or not the tension is appropriate based on the detected difference in the optical flow, that is, a difference between a movement of the tissue and a movement of the treatment tool. When the movement of the tissue is in conjunction with the movement of the treatment tool and the difference in the movements is small, the determination section 114 determines that the tension is low. When the movement of the tissue is not in conjunction with the movement of the treatment tool and the difference in the movements is large, the determination section 114 determines that the tension is high. Note that the tension determination using the optical flow may be combined with the tension determination using the transmittance, the color of the tissue, or changes thereof. With this configuration, it can be expected to increase the accuracy of the tension determination in comparison with the tension determination using only either the optical flow or the transmittance, the color of the tissue, or changes thereof.

As described above, the medical system 10 in the present embodiment includes the treatment tool 310 provided with the end effector 311 that treats the tissue, the imaging device that captures an image of the tissue, and the processor 110. The processor 110 acquires the color information of the target tissue from the captured image output from the imaging device. The target tissue is a portion of the tissue that covers at least a portion of the end effector 311 that has slid into the tissue. The processor 110 estimates a force applied to the target tissue by the end effector 311 from the color information of the target tissue. The processor 110 notifies the operator of a result of the estimation of the force.

In accordance with the present embodiment, at least a portion of the end effector 311 has slid into the tissue as the treatment target, and the force is applied to the tissue that covers at least the portion of the end effector 311 by the end effector 311. The portion of the target tissue to which the force is applied by the end effector 311 is changed in color by the influence of the force. For example, the force is applied to the target tissue and the thickness of the target tissue is changed, whereby the transmittance of the end effector 311 that passes thorough the target tissue is changed. Or the application of the force to the target tissue whitens the target tissue. With this configuration, it is possible to estimate the force applied to the target tissue by the end effector 311 from the color information of the target tissue. By notifying the operator of a result of estimation of the force, it is possible to treat the target tissue while applying an appropriate force to the target tissue with the treatment tool 310 regardless of a level of skill of the operator. With this configuration, it is possible to perform the treatment efficiently and safely.

Additionally, in the present embodiment, the processor 110 recognizes the position of the treatment tool 310 in the captured image, and sets the region of interest AROI to a region corresponding to the end effector 311 in the captured image based on a result of the recognition of the position. The processor 110 recognizes the color information of the target tissue from the image of the region of interest AROI, and estimates the force from the color information of the target tissue.

In accordance with the present embodiment, it is possible to acquire the color information of the target tissue while limiting a target to the region corresponding to the end effector 311 in a field of view in which various objects are seen. By acquiring the color information while limiting the target to a tissue region to which the force is applied by the end effector 311, it is possible to estimate the force with high accuracy.

Additionally, in the present embodiment, the treatment tool 310 includes the shaft 312 connected to the base end of the end effector 311. As shown in FIGS. 6 to 11, the processor 110 recognizes at least one of the end effector 311 or the shaft 312 from the captured image by segmentation, and uses a result of the recognition by segmentation to set the region of interest AROI.

In accordance with the present embodiment, it is possible to recognize at least one of the position of the end effector 311 or the position of the shaft 312 by segmentation, and set the region of interest AROI based on the position. For example, in a case where the end effector 311 is recognized, the region of interest AROI can be set at the recognized position. Alternatively, in a case where only the shaft 312 is recognized, since the position of the end effector 311 hidden under the tissue can be estimated based on the position of the distal end of the shaft 312, the region of interest AROI can be set at the position.

Additionally, in the present embodiment, the region of interest AROI is a range in which the end effector 311 should be seen in the captured image. As described with reference to FIGS. 6 to 9, the processor 110 sets a region surrounded by the edge extension lines LE1 and LE2 that are extended from the edges of the shaft 312 recognized by segmentation and including the end effector 311 as the region of interest AROI.

The end effector 311 mainly exists in the region surrounded by the edge extension lines LE1 and LE2 that are extended from the edges of the shaft 312. By limiting the region of interest AROI to the region surrounded by the edge extension lines LE1 and LE2, it is possible to set the region corresponding to the end effector 311 as the region of interest AROI.

Additionally, in the present embodiment, the processor 110 sets a region surrounded by the edge extension lines LE1 and LE2 and first and second perpendicular lines that are perpendicular to the principal component long-axis vector LC of the shaft 312 as the region of interest AROI. As described with reference to FIGS. 6 and 7, the first perpendicular line is the line LF1 passing through the base end of the end effector 311 or the line LF3 passing through the distal end of the shaft 312. The second perpendicular line is the line LF2 passing through the distal end of the end effector 311.

In accordance with the present embodiment, in a case where the whole of the end effector 311 is seen, the region of interest AROI can be defined by the line LF1 passing through the base end of the end effector 311 and the line LF2 passing through the distal end of the end effector 311. In a case where the base end side of the end effector 311 has slid into the tissue, the region of interest AROI can be defined with use of the line LF3 passing through the distal end of the shaft 312 and the line LF2 passing through the distal end of the end effector 311.

Additionally, in the present embodiment, the first perpendicular line is the line LF3 passing through the distal end of the shaft 312, as described with reference to FIGS. 8 and 9. The second perpendicular line is each of the lines LF4 and LF5 away from the first perpendicular line by a predetermined length toward the distal end side of the end effector 311. The predetermined length may be the fixed length GLA on the image as shown in FIG. 8, or the length GLB that is determined with the shaft diameter SHD serving as a criterion as shown in FIG. 9.

In accordance with the present embodiment, even in a case where the end effector 311 is entirely not seen in the captured image, it is possible to specify the second perpendicular lines with the line LF3 passing through the distal end of the shaft 312 serving as a criterion and define the region of interest AROI using these lines.

Additionally, in the present embodiment, as described with reference to FIG. 11, the processor 110 performs segmentation to recognize a missing region AFKC of the end effector that is missing in the captured image due to overlapping of the end effector 311 with the target tissue and an exposure region AFK of the end effector seen in the captured image. The processor 110 sets the recognized missing region AFKC and the recognized exposure region AFK as the region of interest AROI.

In accordance with the present embodiment, it is possible to set the shape of the end effector 311 itself as the region of interest AROI. Since the color information of the target tissue is obtained from the more limited region, the force applied to the target tissue can be estimated with higher accuracy. Furthermore, since the missing region AFKC of the end effector 311 that has slid into the tissue and that is not seen in the image is recognized, the whole region of the end effector 311 can be set as the region of interest AROI.

Additionally, in the present embodiment, as described with reference to FIGS. 14 and 15, the processor 110 estimates that the force applied to the target tissue by the end effector 311 becomes stronger as the color information of the target tissue becomes closer to the color information of the end effector 311.

Since the color of the target tissue becomes lighter as the force applied to the target tissue becomes stronger, the color of the end effector 311 is transparently seen through the target tissue. That is, as the color information of the target tissue becomes closer to the color information of the end effector 311, it is possible to estimate the stronger force.

Additionally, in the present embodiment, the processor 110 acquires the transmittance of the color of the end effector 311 in the target tissue as an index indicating approximation between the color information of the target tissue and the color information of the end effector 311. The processor 110 estimates that the force applied to the target tissue by the end effector 311 becomes stronger as the transmittance becomes higher.

A degree of transparency of the color of the end effector 311 through the target tissue is expressed by the transmittance. Since the color of the target tissue becomes lighter as the force applied to the target tissue becomes stronger, it is possible to estimate the stronger force as the transmittance becomes higher.

Additionally, in the present embodiment, the processor 110 makes a comparison between the index indicating approximation between the color information of the target tissue and the color information of the end effector 311, and a determination criterion for determination about an index indicating an appropriate force range to determine whether or not the force applied to the target tissue is in the appropriate range. For example, the index is the transmittance, and the determination criterion is the upper limit and lower limit of the transmittance corresponding to the appropriate force range.

The above-mentioned index changes in accordance with the force applied to the target tissue. As a result, it is possible to define a range of the index corresponding to the appropriate force range. With this range serving as the determination criterion, it is possible to determine whether or not the appropriate force is applied to the target tissue.

### 2.3. Second Configuration Example

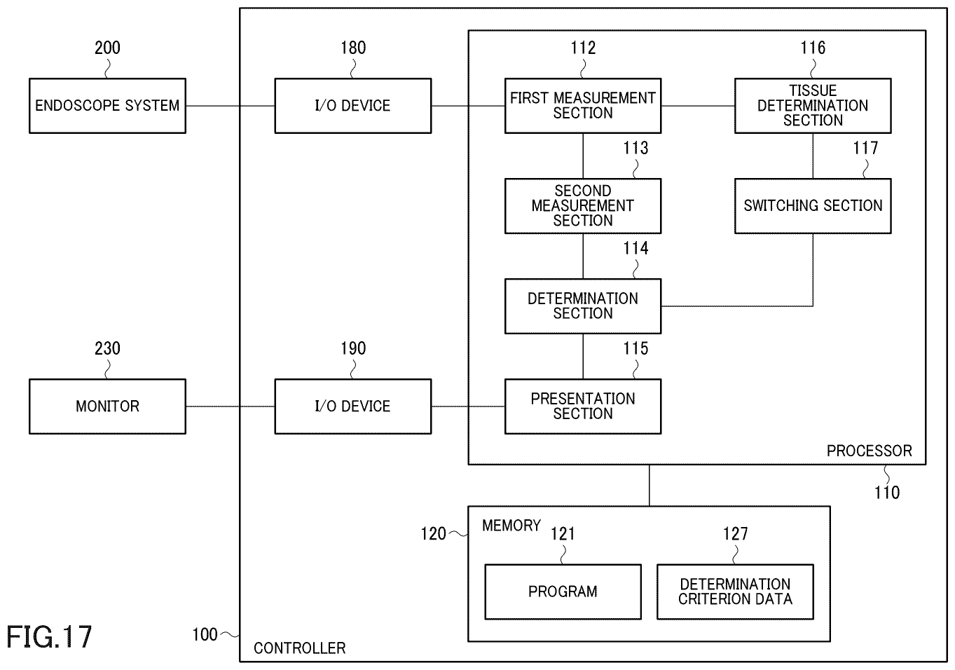

FIG. 17 shows a second configuration example of the controller in the first embodiment. The following description will be mainly given of parts different from the first configuration example, and parts similar to the first configuration example are omitted.

The processor 110 includes a tissue determination section 116 and a switching section 117. The memory 120 stores determination criterion data 127 including a plurality of determination criteria used for the tension determination.

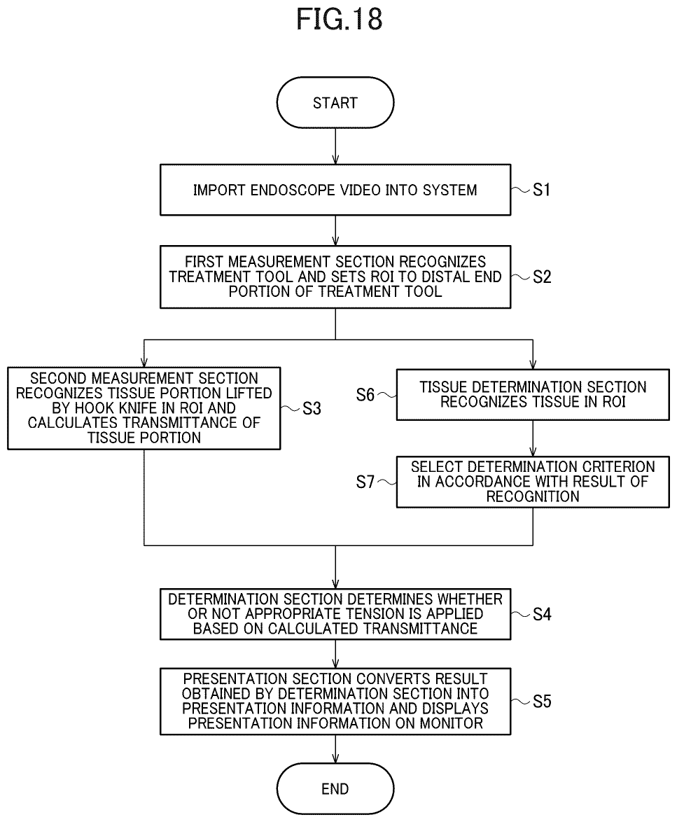

FIG. 18 is a flowchart of processing in the second configuration example. In step S6, the tissue determination section 116 uses the image of the region of interest detected by the first measurement section 112 to recognize a type of a tissue seen in the region of interest. The tissue determination section 116 recognizes whether or not the tissue in the region of interest corresponds to any of a plurality of predetermined tissue types. The tissue type is a type having a possibility of being treated by the hook-knife type monopolar device in an operative method to be executed. Examples of the tissue types include a connective tissue, a serosa, fat, and a small vessel.

In step S7, the switching section 117 selects a determination criterion in accordance with the tissue type recognized by the tissue determination section 116. The determination criterion is a range of transmittance corresponding to the appropriate tension range, and is set with respect to each tissue type.

In step S4, the determination section 114 uses the determination criterion selected by the switching section 117 to determine whether the transmittance calculated by the second measurement section 113 is within the appropriate tension range.

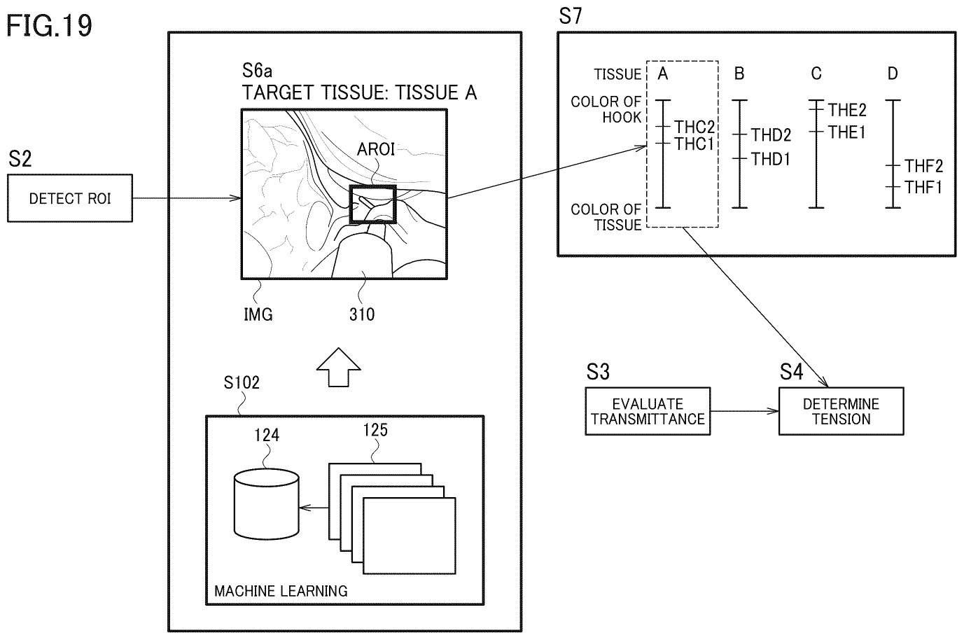

FIG. 19 is a diagram for describing details of processing executed by the tissue determination section and the switching section in the second configuration example.

In step S102, a trained model 124 is generated by machine learning. This step S102 is executed in the training phase, and may not be included in step S6 in FIG. 18 in the inference phase. Training data 125 includes the image of the region of interest in a plurality of endoscope images and an annotation added to each image of the region of interest. Object detection mainly performed by segmentation is assumed, and the annotation is data indicating a region of the tissue in the image of the region of interest. Specifically, the annotation of the type of each tissue seen in the region of interest and a range of the tissue is added to the image of the region of interest. The machine learning is executed by the training device such as the information processing device. The processor of the training device inputs an endoscope image of the training data 125 to a training model, which detects the region of the treatment tool from the endoscope image, and updates a weight parameter of the training model based on an error between a result of the detection and the annotation of the training data 125. The training device repeats this updating processing on multitudes of endoscope images to generate the trained model 124.

In step S6a, the tissue determination section 116 inputs the image of the region of interest AROI to the trained model 124, and the trained model 124 detects a region of each tissue from the image of the region of interest AROI. The tissue determination section 116 determines overlapping of the detected region of each tissue and the hook detected by the first measurement section 112, and thereby determines a tissue type of the tissue lifted by the hook. Assume that the tissue lifted by the hook is a tissue A among tissues A, B, C, and D.

In step S7, the switching section 117 selectness a determination criterion for the determined tissue A among determination criteria for the tissues A, B, C, and D. Specifically, a lower limit of transmittance with respect to the tissue A is THC1, and an upper limit thereof is THC2. The lower limit of transmittance with respect to the tissue B is THD1, and an upper limit thereof is THD2. A lower limit of transmittance with respect to the tissue C is THE1, and an upper limit thereof is THE2. A lower limit of transmittance with respect to the tissue D is THF1, and an upper limit thereof is THF2. The switching section 117 selects the lower limit THC1 and lower limit THC2 of the transmittance with respect to the tissue A.

Note that in step S6a, the tissue determination section 116 may perform segmentation with respect to the whole of the endoscope image IMG. In this case, training data of machine learning includes an endoscope image, a name of the tissue and a region of the tissue attached to each endoscope image. Additionally, the endoscope image IMG acquired in step S1 and information about the region of the treatment tool recognized in step S2 are input to the tissue determination section 116.

In step S6a, the tissue determination section 116 may recognize the tissue type of the tissue lifted by the hook directly from the endoscope image IMG. In this case, the training data of machine learning includes the endoscope image and the name of the tissue added to each endoscope image. The name of the tissue is a name of the tissue lifted by the hook in the endoscope image of the training data.

Additionally, in the calculation of the transmittance in step S3, the color of the tissue serving as the criterion may be preliminarily set instead of being acquired from the image. In this case, in step S7, the switching section 117 may select the color of the tissue and the range of the transmittance depending on the tissue type recognized in step S6a. In step S3, the second measurement section 113 may use the selected color of the tissue to calculate the transmittance.

As described above, in the medical system 10 in accordance with the present embodiment, the processor 110 recognizes the type of the target tissue from the image of the region of interest AROI or the endoscope image. The processor 110 selects the determination criterion corresponding to the recognized type from the plurality of determination criteria corresponding to a plurality of tissue types. The processor 110 uses the selected determination criterion to determine whether or not the force applied to the target tissue is in the appropriate range.

How much force should be applied during a treatment is different depending on the tissue type. In accordance with the present embodiment, it is possible to select the determination criterion depending on the tissue type of the tissue as the treatment target of the treatment tool 310, whereby it becomes possible to select the determination criterion for an optimum force depending on the tissue type. With this configuration, it is possible to perform a treatment such as incision with the treatment tool such as the hook knife more safely in comparison with a case using a single determination criterion.

### 2.4. Third Configuration Example

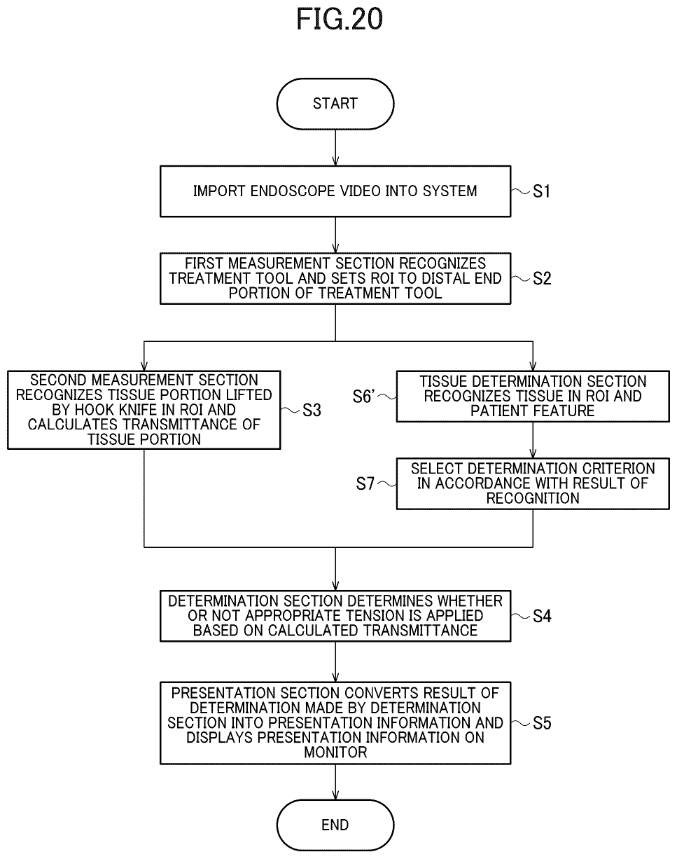

FIG. 20 is a flowchart of processing in a third configuration example. Note that a configuration example of the controller 100 is similar to that in the second configuration example shown in FIG. 17. The following description will be mainly given of parts different from the first and second configuration examples, and parts similar to the first and second configuration examples are omitted.

In step S6′, the tissue determination section 116 uses the image of the region of interest detected by the first measurement section 112 and patient information to recognize the tissue type of the tissue seen in the region of interest and features of a patient. The patient information is information indicating the features of the patient as the treatment target. Examples of the patient information include the patient's weight, height, body mass index (BMI), gender, age, medical history, and the like.

In step S7, the switching section 117 selects the determination criterion in accordance with the tissue type and the features of the patient that have been recognized by the tissue determination section 116.

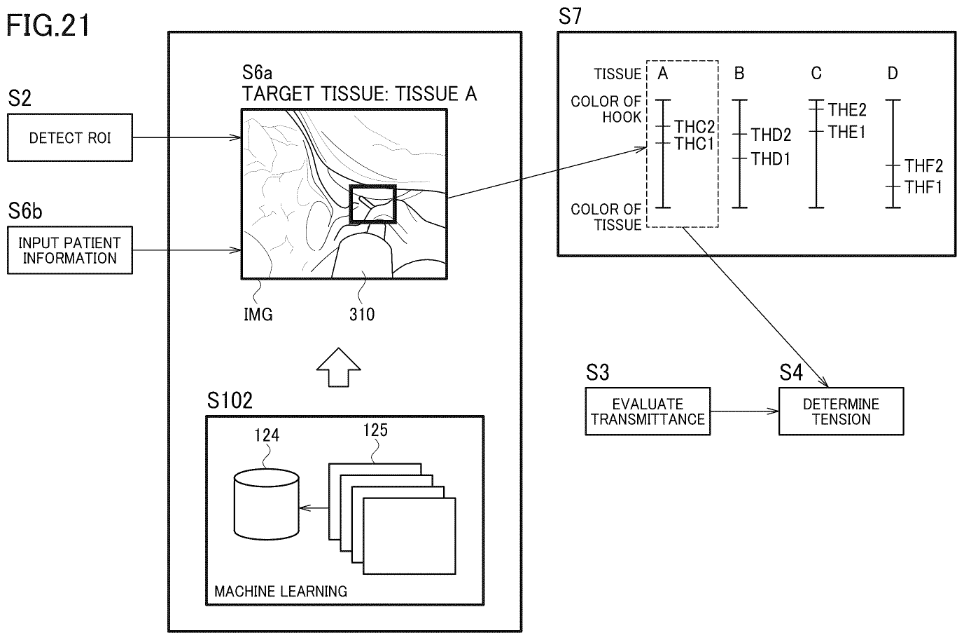

FIG. 21 is a diagram for describing details of processing executed by the tissue determination section and the switching section in the third configuration example.

In step S102, the training data 125 includes an image of the region of interest in a plurality of endoscope images, an annotation image added to each image of the region of interest, and the patient information regarding the patient in each endoscope image. The annotation is data indicating a region of the tissue in the image of the region of interest. Since features of the tissue such as a color and an appearance are different depending on the features of the patient, machine learning using the patient information is performed, whereby segmentation with higher accuracy can be implemented.

In step S6b, the patient information is input to the tissue determination section 116. For example, the patient information may be input to the medical system by the operator or the like, or may be input to the medical system from an upper level system such as an electronic health record system. In step S6a, the tissue determination section 116 inputs the image of the region of interest AROI and the patient information to the trained model 124, and the trained model 124 detects the region of each tissue from the image of the region of interest AROI. The tissue determination section 116 determines overlapping of the detected region of each tissue and the hook detected by the first measurement section 112, and thereby determines the tissue type of the tissue lifted by the hook. Assume that the tissue lifted by the hook is the tissue A among the tissues A, B, C, and D.

In step S7, the switching section 117 selects the determination criterion for the tissue A among a determination criterion set corresponding to the features of the patient based on a result of the determination about the tissue and the features of the patient. The lower limits THC1 to THF1 of the transmittance and the upper limits THC2 to THF2 of the transmittance are defined with respect to each tissue, but lower limit values and upper limit values are different depending on the features of the patient.

Note that in the calculation of the transmittance in step S3, the color of the tissue serving as the criterion may be preliminarily set instead of being acquired from the image. In this case, in step S7, the switching section 117 may select the color of the tissue and the range of the transmittance depending on the tissue type and the features of the patient recognized in step S6a. In step S3, the second measurement section 113 may use the selected color of the tissue to calculate the transmittance.

Since properties of the tissue as the treatment target tissue are different depending on the features of the patient, there is a possibility that the appropriate force range is different. In accordance with the third configuration example, it is possible to select the determination criterion for determination about the appropriate force depending on the tissue type and the features of the patient. With this configuration, it is possible to perform a treatment such as incision using the treatment tool such as the hook knife more safely in comparison with a case of using a single determination criterion or a case of not using the features of the patient.

### 3. Second Embodiment

In a second embodiment, when a treatment is performed with a monopolar treatment tool in laparoscopic surgery, the medical system automatically adjusts output of the monopolar treatment tool in accordance with a tension applied to the treatment target tissue. Note that the monopolar treatment tool is not limited to the hook-knife type monopolar device, but may be a spatula scalpel or the like. Additionally, the second embodiment is not limited to the case where the tension is applied to the treatment target tissue by the monopolar treatment tool like the hook-knife type monopolar device, but the tension may be applied to the treatment target tissue by forceps or the like other than the monopolar treatment tool. Note that the second embodiment may be combined with the first embodiment.

There is an issue that a non-expert physician is unaccustomed to adjusting the tension depending on the tissue in comparison with an expert physician. For example, in a tissue in which emphasis is placed on incision such as a thin membrane or a connective tissue, it takes time to perform incision in a case where a tension is insufficient with respect to output of the monopolar treatment tool. Since a range of thermal invasion expands when it takes time to perform incision, there is a possibility that thermal invasion extends to peripheral important tissues or important organs. Or in a case where the tension is excessive with respect to the output of the monopolar treatment tool, there is a possibility that coagulation in an incised portion becomes insufficient and bleeding occurs.

In accordance with the present embodiment, the output of the monopolar treatment tool is automatically adjusted to be appropriate output with respect to the tension applied to the treatment target tissue. With this configuration, it is possible to perform the treatment in combination of the appropriate tension and the output regardless of a level of skill of a physician. For example, also in a case where the tension applied to the tissue in which emphasis is placed on incision is insufficient, the output is automatically adjusted, whereby it becomes possible to perform incision efficiently. Alternatively, also in a case where the tension applied to the tissue in which emphasis is placed on coagulation is excessive, the output is automatically adjusted, whereby it becomes possible to infallibly perform hemostasis and coagulation.

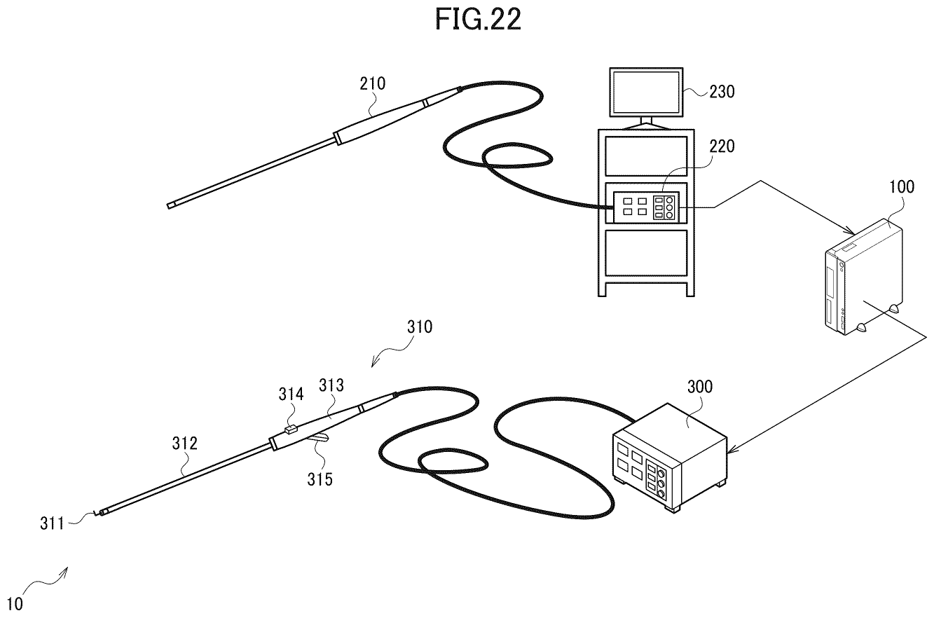

FIG. 22 shows a configuration example of the medical system in accordance with the second embodiment. The following description will be mainly given of parts different from the configuration example of the medical system in the first embodiment described with reference to FIG. 2, and parts similar to the first embodiment are omitted. In the second embodiment, the treatment tool 310 is the monopolar treatment tool. There are various shape types of an end effector of the monopolar treatment tool. Examples of the monopolar treatment tool include the hook-knife type monopolar device and the spatula scalpel.

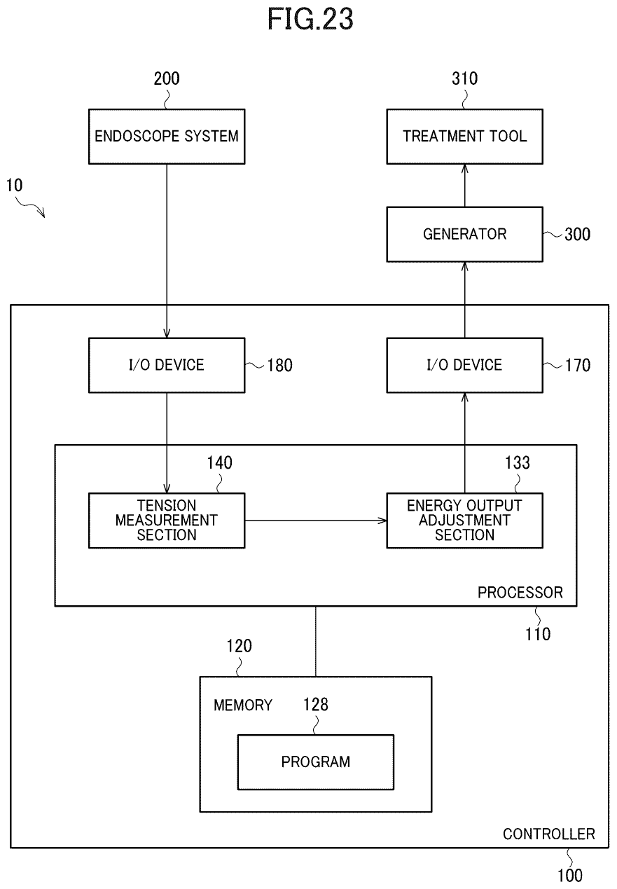

The controller 100 measures the tension applied to the treatment target tissue from the endoscope image, and adjusts an output setting of the generator 300 in accordance with the measured tension. FIG. 23 shows a first configuration example of the controller in accordance with the second embodiment. The controller 100 includes the I/O device 180 that receives the endoscope image from the endoscope system, the processor 110, the memory 120, and an I/O device 170 that outputs an output setting output from the processor 110 to the generator 300. The memory 120 stores a program 128 in which contents of processing performed by a tension measurement section 140 and an energy output adjustment section 133 are described. The processor 110 executes the program 128 to execute processing of each of the tension measurement section 140 and the energy output adjustment section 133.

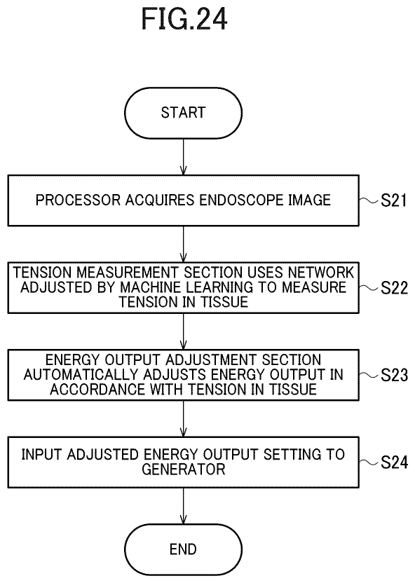

FIG. 24 is a flowchart of processing in the first configuration example in accordance with the second embodiment. In the step S21, the processor 110 acquires an endoscope image from the endoscope system 200 via the I/O device 180.

In step S22, the tension measurement section 140 uses a trained model obtained by machine learning to measure a tension in the tissue from the endoscope image.

In step S23, the energy output adjustment section 133 adjusts an output setting in accordance with the measured tension. The output setting to be adjusted is an output level, an output type, or both of the output level and the output type. The output type is cutting, coagulation, or a blend of cutting and coagulation. In a case where the tension is insufficient with respect to the present output setting in the tissue in which emphasis is placed on incision such as a thin membrane or a connective tissue, the energy output adjustment section 133 increases an output level for cutting. in a case where the tension is excessive with respect to the present output setting in the tissue in which emphasis is placed on coagulation such as fat, a small vessel, and a lymph vessel, the energy output adjustment section 133 decreases the output level for cutting. The output level includes voltage, current, and power. Alternatively, in a case of output for coagulation, a duty ratio may be changed as described later.

Alternatively, in a case where the treatment tool 310 capable of producing blended output, the energy output adjustment section 133 may change contribution of cutting in accordance with the tension. The blended output is output by which the tissue can be cut while being coagulated. Contribution of coagulation and cutting can be changed by a change of a duty ratio of a high-frequency pulse. The duty ratio is, for example, a time ratio between ON and OFF at an ON/OFF cycle of the high-frequency pulse or a time ratio between output for incision and output for coagulation. By increasing ON in the time ratio or the output for incision in the time ratio, it is possible to increase the contribution of cutting. Conversely, by decreasing ON in the time ratio or the output for incision in the time ratio, it is possible to decrease the contribution of cutting. In a case where the tension is insufficient with respect to the present output setting in the tissue in which emphasis is placed on incision such as a thin membrane or a connective tissue, the energy output adjustment section 133 increases the contribution of cutting. In a case where the tension is excessive with respect to the present output setting in the tissue in which emphasis is placed on coagulation such as fat, a small vessel, and a lymph vessel, the energy output adjustment section 133 decreases the contribution of cutting. Alternatively, the above-mentioned adjustment of the output level may be performed in combination. Additionally, the adjustment of the output level may be performed only before the start of energy output, or may be sequentially performed in accordance with a tension measured also during output as described later.

In the step S24, the energy output adjustment section outputs the adjusted energy output setting to the generator 300. The generator 300 drives the treatment tool 310 with set energy output.

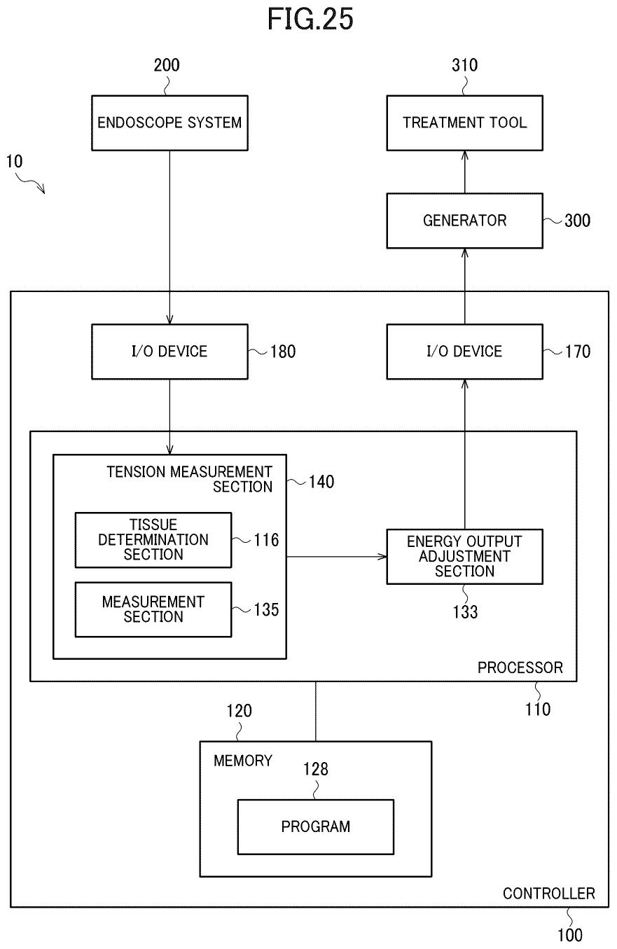

FIG. 25 shows the second configuration example of the controller in accordance with the second embodiment. In this example, the controller 100 recognizes a type of the treatment target tissue from the endoscope image, measures a tension applied to the treatment target tissue, and adjusts an output setting of the generator 300 in accordance with the measured tension. The tension measurement section 140 includes the tissue determination section 116 and a measurement section 135. The description herein is given of the tissue determination section 116 and the measurement section 135 in a case where the treatment tool 310 is the spatula scalpel as one example.

The tissue determination section 116 uses a trained model to recognize the treatment target tissue from an endoscope image. The trained model has been machine-learned with an endoscope image, a label indicating a proximity state between a monopolar device and a tissue in the endoscope image, and a label indicating a tissue to which the monopolar device is in proximity as training data. The proximity state includes a state of proximity, a state of contact, and a state of non-contact.

The measurement section 135 evaluates a tension in the treatment target tissue based on color information of a tissue around the distal end of the treatment tool. For example, since the tissue becomes more tinged with white due to ischemia in the tissue or the inflow of the air into between layers as the tension becomes higher, HSV color space coordinates become closer to those of a white region. The measurement section 135 has, for example, the following configuration. Information about the treatment target region recognized by the tissue determination section 116 is input to the measurement section 135, and the measurement section 135 may acquire the color information from the treatment target region. Alternatively, an endoscope image is input to the measurement section 135, and the measurement section 135 may recognize the color information from the endoscope image. Alternatively, the measurement section 135 may use a method similar to that used by the first measurement section and the second measurement section in accordance with the first embodiment to recognize an image of the spatula scalpel from the endoscope image as the region of interest and acquire the color information of the recognized region of interest. In this case, the tissue determination section 116 may recognize the treatment target region from the whole of the endoscope image or may recognize the treatment target region from the image of the region of interest in the endoscope image.

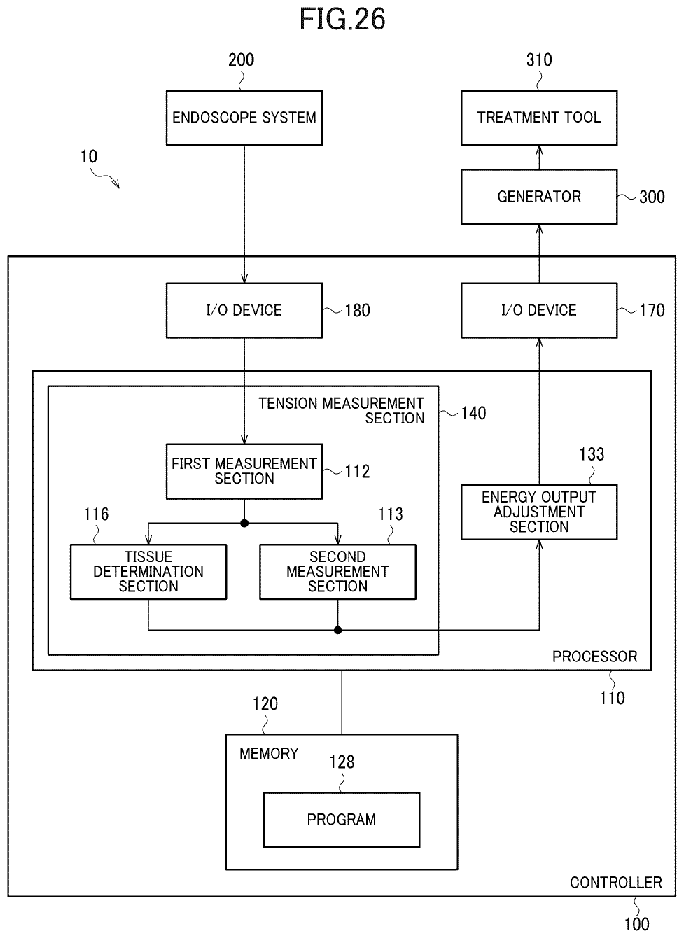

FIG. 26 shows a third configuration example of the controller in the second embodiment. In this example, the tension measurement section 140 includes the first measurement section 112, the second measurement section 113, and the tissue determination section 116, which are similar to those in the first embodiment. The description herein is given of an example in which the treatment tool 310 is the hook-knife type monopolar device, which is similar to that in the first embodiment.

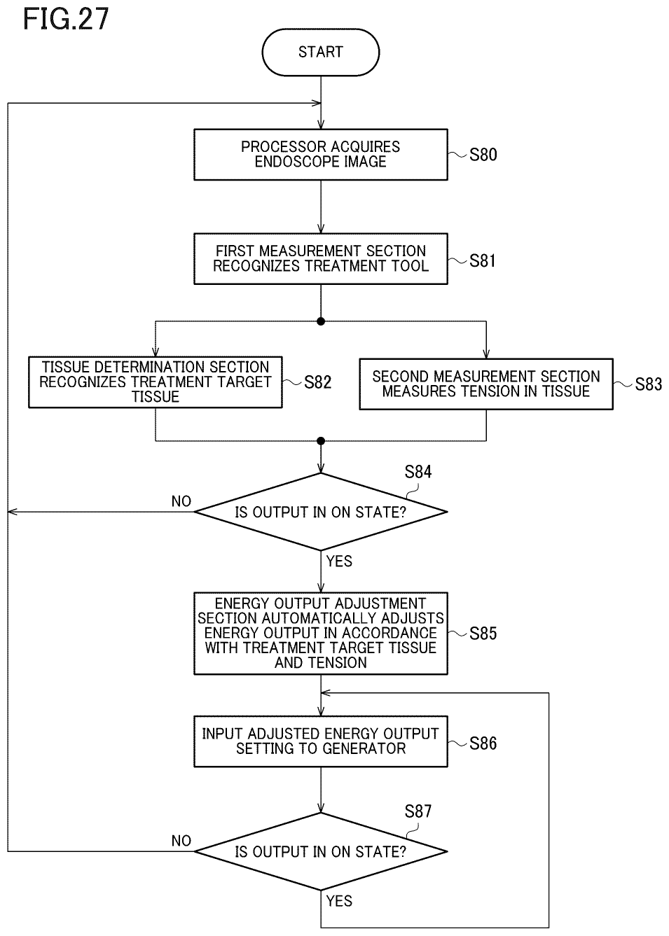

FIG. 27 is a first flowchart of processing in the third configuration example in the second embodiment. In step S80, the processor acquires an endoscope image from the endoscope system.

In step S81, the first measurement section 112 performs image recognition using machine learning to detect a region in the vicinity of the distal end of the monopolar treatment tool as the region of interest. The recognition method is as described in the first embodiment.

In step S82, the tissue determination section 116 determines a tissue as a treatment target of the monopolar treatment tool. The method of determining the tissue is as described in the first embodiment.

In step S83, the second measurement section 113 calculates transmittance of the hook from an image of the region of interest, and evaluates a tension in the treatment target tissue based on the transmittance. The method of evaluating the tension is as described in the first embodiment.

In step S84, the processor 110 determines whether or not output of the monopolar treatment tool is in an ON state. In a case where the output is not in the ON state, step S80 is executed. In a case where the output is in the ON state, in step S85, the energy output adjustment section 133 adjusts the output to a cutting output level in accordance with the measured tension.

In step S86, the energy output adjustment section outputs an adjusted energy output setting to the generator 300. The generator 300 drives the monopolar treatment tool with set energy output.

In step S87, the processor 110 determines whether or not the output from the monopolar treatment tool is in the ON state. In a case where the output is not in the ON state, step S80 is executed. In a case where the output is in the ON state, step S86 is executed. That is, after the energy setting is automatically adjusted at the start of output in step S85, step S86 is performed in a loop while the output is in the ON state, so that the energy setting is not automatically adjusted.

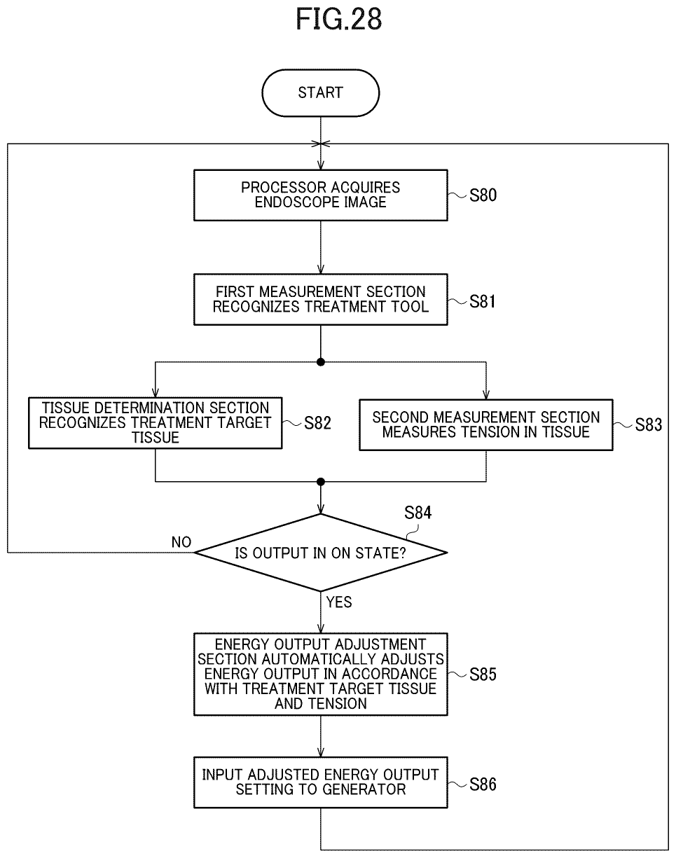

FIG. 28 is a second flowchart of processing in the third configuration example in the second embodiment. In this configuration, step S87 is omitted from the flowchart of FIG. 27, and the processing returns to step S80 after step S86. In this flow, since steps S80 to S86 are performed in a loop while the output is in the ON state, the energy setting is automatically adjusted not only at the start of output but also during the output.

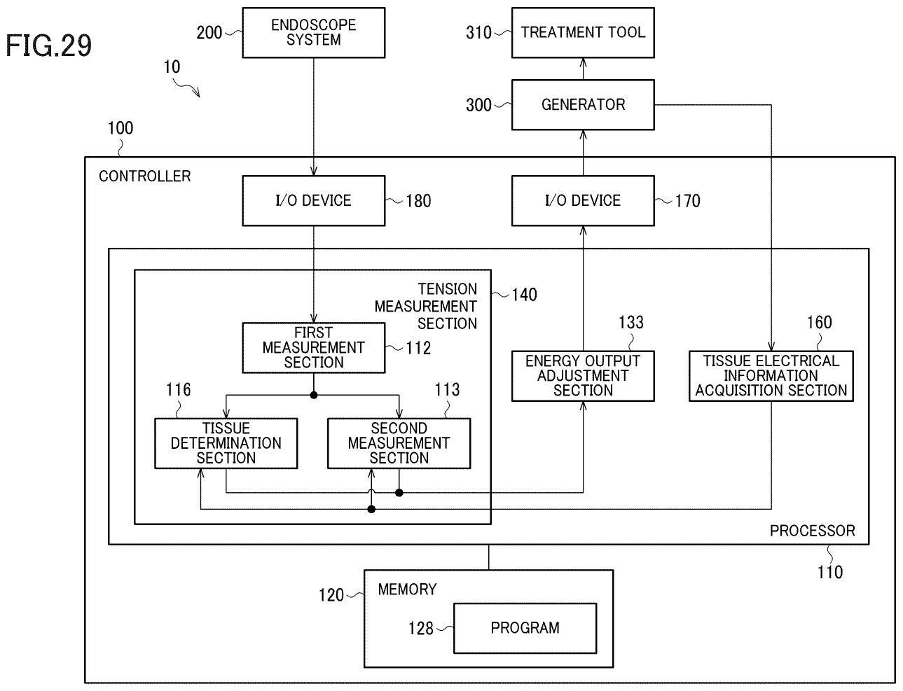

FIG. 29 shows a fourth configuration example of the controller in the second embodiment. In this configuration example, the processor 110 further includes a tissue electrical information acquisition section 160. The description herein is given of an example in which the tissue electrical information acquisition section 160 is combined with the third configuration example in FIG. 26, but the tissue electrical information acquisition section 160 may be combined with the second configuration example in FIG. 25 so that the tissue determination section 116 and the measurement section 135 are configured to use the output of the tissue electrical information acquisition section 160.

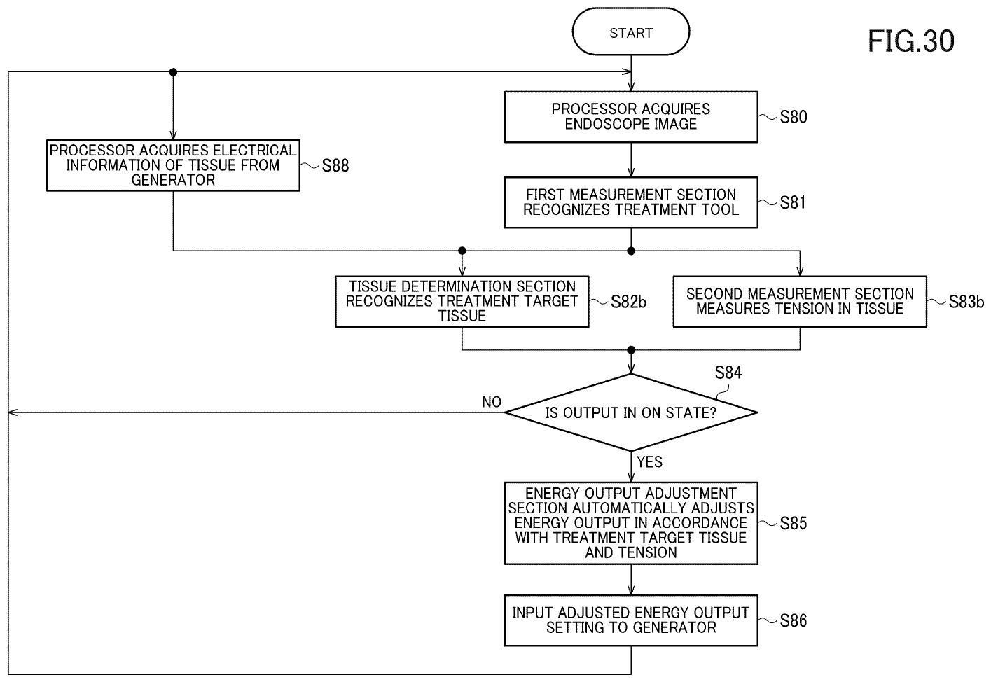

FIG. 30 is a flowchart of processing in the fourth configuration example in the second embodiment. Step S88 is added to the flow in FIG. 28. In this configuration, step S88 is performed in parallel with steps S80 and S81, and in a case where the output is in an OFF state in step S84 or after step S86, the processing returns to steps S88 and S80. Additionally, in the configuration, a result of processing in step S88 is used in steps S82b and S83b.

In step S88, the tissue electrical information acquisition section 160 acquires electrical information of the tissue from the generator 300. The electrical information includes current or voltage after the start of the output, impedance or power obtained from the current or the voltage, an accumulated amount or a change amount obtained from elementary calculation of the current, the voltage, the impedance, or the power, and a freely selected parameter that combines some of the current, the voltage, the impedance, the power and the change amount.

In step S82b, the tissue determination section 116 recognizes the treatment target tissue of the monopolar treatment tool, and corrects a result of recognition of the tissue in accordance with the electrical information obtained by the generator 300. For example, in a case where the impedance is low, there is a possibility that the treatment target tissue is a thin membrane, a connective tissue, a small vessel, or a lymph vessel. For example, in a case where the impedance is high, there is a possibility that the treatment target tissue is fat or a thick membrane tissue.

In step S83b, the second measurement section 83 evaluates the tension in the treatment target tissue based on the transmittance of the hook, and corrects a result of evaluation of the tension in accordance with the electrical information obtained by the generator 300. For example, since the impedance of the tissue changes depending on the high/low of the tension, the second measurement section 83 uses the change of the impedance to correct the result of evaluation of the tension. Note that only either the tissue determination section 116 or the second measurement section 83 may be configured to perform correction using the electrical information.

As described above, the medical system 10 in accordance with the present embodiment includes the treatment tool 310 provided with the end effector that treats the tissue, the generator 300 that adjusts energy output from the end effector, the imaging device that captures an image of the tissue, and the processor 110. The processor 110 performs first processing of estimating a force applied to the tissue to be treated by the treatment tool 310 from the captured image and changing the output setting of the generator 300 based on the result of estimation of the force.

There is a case where a non-expert physician or the like cannot implement an appropriate tension when performing a treatment on the tissue. In accordance with the present embodiment, the force applied to the tissue is estimated and the output setting appropriate for the force is automatically changed. With this configuration, it is possible to perform the treatment in combination of the appropriate tension and the output regardless of a level of skill of the operator.

Additionally, in the present embodiment, the processor 110 may acquire the color information of the target tissue from the captured image output from the imaging device. The target tissue is a portion of the tissue that covers at least a portion of the end effector 311 that has slid into the tissue. The processor 110 may estimate the force applied to the target tissue by the end effector 311 from the color information of the target tissue. The processor 110 may change the output setting of the generator 300 based on a result of estimation of the force.

With this configuration, it is possible to estimate the force applied to the target tissue from the color information of the target tissue using a method similar to that in the first embodiment. The method can be applied to, for example, a case where the treatment tool 310 is the hook-knife type monopolar device or other cases.

### 4. Third Embodiment

In a third embodiment, the medical system recognizes the treatment tissue and also recognizes a metal around the energy device when the energy device is used in endoscopic surgery. The medical system notifies the operator of abnormal proximity between the metal and an electrode of the energy device, and also brings the energy device into a state of being unable to output energy. In a case where the operator rejects the stop of output of the medical system, the medical system automatically restricts selectable energy sequences depending on a probability of estimation of abnormal proximity, and selects an energy sequence from the restricted options. Note that the energy device may be a monopolar device, a bipolar device, an ultrasound wave device, a high frequency/ultrasound combination device, or the like. Note that the third embodiment may be combined with the first embodiment or the second embodiment.

With heat diffusion to an unintended part or an unintended range via a staple of a stapler or forceps in the vicinity of the energy device, there is a possibility that a thermal burn occurs on an important tissue in proximity to the metal. For this reason, in a case where a treatment is performed on a membrane or the like in the vicinity of a tissue that requires a sealing force or in proximity to an important tissue, it is an unfavorable operation to perform a treatment in the vicinity of the metal. When there is a possibility that such an unfavorable operation is executed, a system that makes notification to the operator, or a system that prevents execution of the unfavorable operation is desirable.

A configuration of the medical system is similar to the configuration example in the second embodiment shown in FIG. 22. In the third embodiment, the controller 100 detects the treatment target tissue and the metal from the endoscope image, and detects abnormal proximity between the metal and the electrode of the energy device. The controller 100 notifies the operator of the abnormal proximity and also gives an instruction for the stop of energy output to the generator 300. The controller 100 includes the I/O device that receives an endoscope image from the endoscope system, the processor, the memory, and the I/O device that outputs an output setting output from the processor to the generator 300. The memory stores a program in which contents of processing performed by each of a tissue detection section, a distance-measurement section, a device detection section, and a determination section. The processor executes the program to execute the processing of each of the tissue detection section, the distance-measurement section, the device detection section, and the determination section.

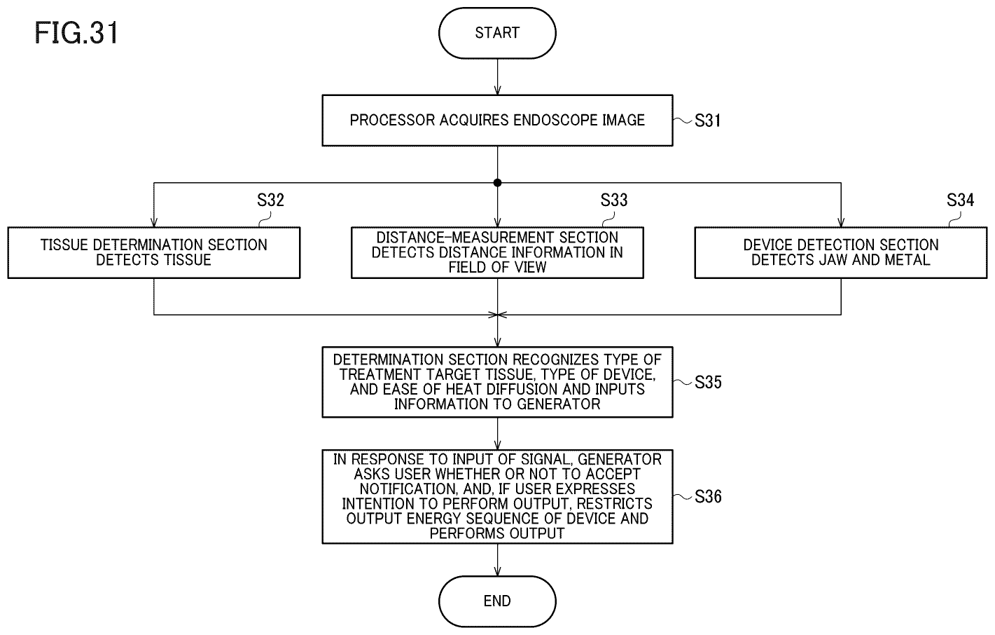

FIG. 31 is a flowchart of processing in the third embodiment. In the step S31, the processor acquires an endoscope image from the endoscope system.

In step S32, the tissue detection section detects a tissue from an endoscope image. The tissue detection section recognizes a region of each tissue seen in the endoscope image by, for example, segmentation using machine learning.

In step S34, the device detection section detects the end effector of the energy device and the metal from the endoscope image. The end effector is, for example, a bipolar device or a jaw of a combination device, but is not limited thereto and may be an electrode of a monopolar device or the like. The device detection section, for example, performs segmentation using machine learning to recognize the region of the end effector and the region of the metal seen in the endoscope image.

In step S33, the distance-measurement section detects distance information within a field of view. The distance information includes a distance between the end effector of the energy device and the metal, a distance between the metal and the important tissue, or a distance between the end effector and the important tissue. The distance-measurement section, for example, uses a result of detection performed by the tissue detection section and the device detection section to detect the distance information. The distance-measurement section may calculate the distance with a geometric operation, recognize the distance using machine learning, or replace the distance with the number of separated pixels on the image.

In step S35, the determination section recognizes a type of the treatment target tissue, a type of the energy device, and ease of heat diffusion from the energy device based on a result of the detection in steps S32 to S34. The determination section recognizes abnormal proximity between the end effector and the metal to estimate a risk for a thermal burn, and outputs setting information corresponding to the risk for a thermal burn to the generator 300. Specifically, machine learning is performed with an endoscope image and label data added to the endoscope image serving as training data. The label data is a type of an organ or a tissue, a type of a surgical instrument, distance information, and the whole or part of thermal burn distance information as a treatment result. The thermal burn distance information indicates a distance on which the thermal burn affects as the treatment result, and indicates, for example, a distance between the end effector to the end of a range of the thermal burn. The determination section uses the trained model to estimate the risk for a thermal burn based on a result of detection in steps S32 to S34. In a case where the risk for a thermal burn is outside a permissible range, the determination section outputs information indicating that energy output is not favorable to the generator 300. Note that various patterns can be assumed about what kind of information is used by the determination section to estimate the risk for a thermal burn. For example, the determination section may recognize the abnormal proximity between the end effector and the metal only from the distance-measurement information. In this case, the label data used in machine learning may be only the distance-measurement information. Alternatively, the determination section may recognize the abnormal proximity between the end effector and the metal from a combination of the type of the organ or the tissue, the type of the surgical instrument, the distance-measurement information, and a plurality of pieces of thermal burn distance information or the whole of the thermal burn distance information as the treatment result. In this case, the label data used in machine learning is only required to include information used for recognition performed by the determination section.

In step S36, in a case where unfavorable information is input from the determination section, the generator 300 notifies the operator of the energy output being unfavorable. Examples of the notification include monitor display, a sound, and vibrations. In addition, the generator 300 accepts, from the operator, input indicating whether or not the operator has an intention to perform energy output in response to the notification about a state of unfavorable energy output. In a case where the operator expresses the intention to perform energy output, the generator 300 performs energy output from the energy device. In a case where a probability of estimation for the proximity of the metal or the risk for a thermal burn is high, the generator 300 may perform output in a restricted energy sequence. For example, the generator 300 causes only an energy sequence for a low temperature burn to be selectable. In a case where the operator rejects the notification about the state where the energy output is unfavorable, the generator 300 performs energy output in the energy sequence for a low temperature burn. The energy sequence for a low temperature burn is, for example, an energy sequence for outputting ultrasound waves alone in the high frequency/ultrasound combination device. Note that an energy sequence that is selectable or used to perform output is appropriately notified to the operator so that the operator can recognize the energy sequence. The energy sequence is generally displayed on the generator 300, but another method may be adopted such as display on a screen of an endoscope as described later.

As described above, the medical system 10 in accordance with the present embodiment includes the treatment tool 310 provided with the end effector that treats the tissue, the generator 300 that adjusts energy output from the end effector, the imaging device that captures an image of the tissue, and the processor 110. The processor 110 performs second processing of detecting the metal at the periphery of the end effector from the image captured by the imaging device and changing the output setting of the generator 300 based on a result of the detection of the metal.

In a case where there is the metal such as a staple of a stapler and forceps at the periphery of the end effector of the treatment tool that treats the tissue with energy output, the risk for a thermal burn increases in peripheral tissues or peripheral organs via the metal. In accordance with the present embodiment, in a case where the metal is detected from the periphery of the end effector and the risk for a thermal burn is high, it is possible to change the output setting of the generator 300 so as to decrease the risk for a thermal burn. With this configuration, the method of applying energy that can prevent thermal invasion on the surroundings regardless of whether or not the operator has recognized the surrounding metal. Additionally, the operator may be further notified of the presence of a proximity metal. With this configuration, it is possible to warn the operator of the presence of the proximity metal regardless of whether or not the operator has recognized the surrounding metal.

### 5. Fourth Embodiment

Even if determination made by AI is introduced into a medical device, sensitivity or specificity of the determination is about 90%. For this reason, a state where the determination made by the system can be completely trusted has not been reached. Even if the AI is introduced into a treatment using the energy device and an energy setting optimum for the tissue is determined by the AI, there is a case where the energy setting is inadequate as described above. Meanwhile, obtaining approval for the determination made by the AI from the operator is conceivable in order to guarantee safety. However, if an operation to obtain approval from the operator is performed every time an energy change is made by the AI, usability decreases.

In the present embodiment, it is possible to propose a solution to compatibility between the guarantee of safety by intervention by the operator's determination and the operability of the energy treatment tool. Note that the fourth embodiment may be combined with the first embodiment, the second embodiment, or the third embodiment.

The medical system in the present embodiment has a configuration (i), (ii), or (iii).
(i) A mechanism for inputting approval for a change to the energy setting from the operator and a mechanism for inputting an instruction for energy output are identical. In a case of trusting a result of determination made by the system, the operator presses the mechanism for inputting the approval and the output. In a case of rejecting the result of determination, the operator presses a mechanism for inputting an instruction for normal output.
(ii) Different output sounds are allocated to respective energy settings, and after an energy application button is pressed, only a beep sound is emitted without energy output for a short amount of time.
(iii) The system performs scene recognition, and presents a basic setting in a recognized scene to the operator. The operator gives approval in a case where the presentation is appropriate for a main operation in the scene. The system makes a request for approval to the operator only when a tissue that is different from a target tissue of the main operation is gripped during a treatment. If the operator gives approval, the energy setting is changed, and the treatment is performed.

With the above-mentioned configurations, the following effects can be obtained.
(i) The mechanism for inputting approval from the operator and the mechanism for inputting the output being identical enables an energy treatment by a single touch without an additional operation for approval. Additionally, when the operator cannot trust an AI-recommended setting, it is also possible to make a normal energy treatment setting, and freely select an energy setting that the operator thinks is safe.
(ii) The operation of the energy treatment is not different from that of an existing treatment tool. However, since there is a time lag from the pressing of the energy application button to the application of energy, the operator can become aware of a difference from energy that is assumed by the operator during this time lag, and stop the energy output.
(iii) By making the energy setting suitable for the main tissue or the state, the AI-recommended setting is not changed until the main tissue or the state changes. With this configuration, it is possible to reduce the number of the operations for approval itself, the operations being performed by the operator to give approval to the energy change.

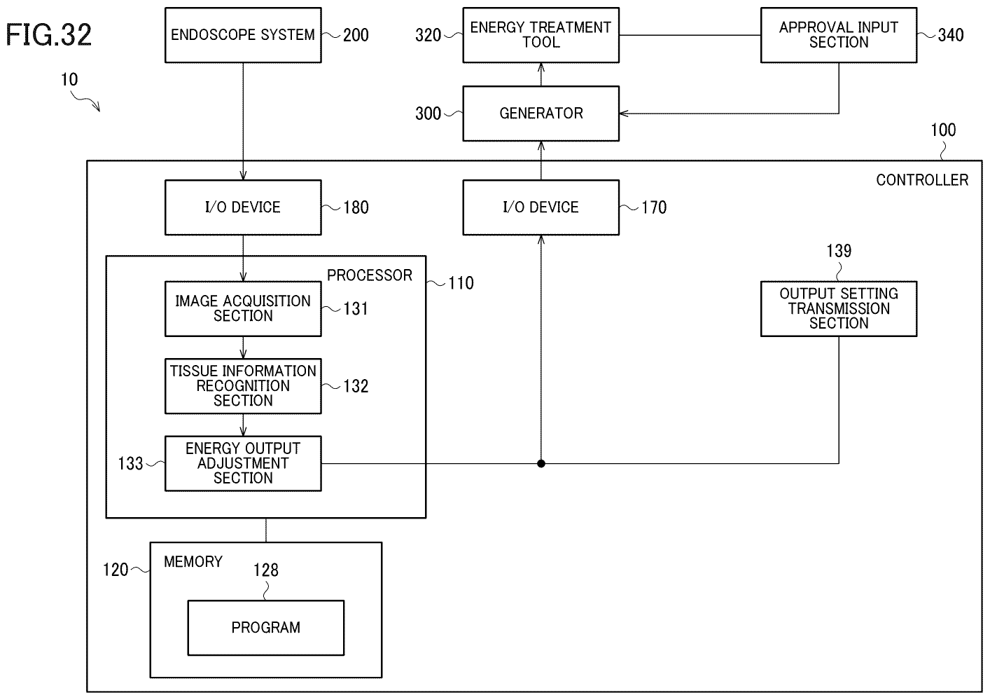

FIG. 32 is a configuration example of a medical system and a controller in a fourth embodiment. The medical system 10 includes the endoscope system 200, the controller 100, the generator 300, an energy treatment tool 320, an approval input section 340.

The endoscope system 200 corresponds to, for example, the endoscope 210, the main body device 220 of the endoscope, and the monitor 230 described with reference to FIG. 2.

The energy treatment tool 320 may be an energy device of various kinds, and may be, for example, a monopolar device, a bipolar device, an ultrasound device, a high frequency/ultrasound combination device, or the like.

The approval input section 340 is an operation mechanism used by the operator to input approval, and may be a button, a switch, a dial, a touch panel, or the like. The approval input section 340 may be integrally provided with the energy treatment tool 320, or may be provided as a device different from the energy treatment tool 320.

The controller 100 includes the I/O device that receives an endoscope image from the endoscope system, the processor, the memory, the I/O device that outputs an output setting output from the processor to the generator 300, and an output setting transmission section 139. The memory stores the program 128 in which contents of processing performed by each of an image acquisition section 131, an tissue information recognition section 132, and the energy output adjustment section 133 are described. The program 128 may include a trained model obtained by machine learning. The processor executes the program 128 to execute processing of each of the image acquisition section 131, the tissue information recognition section 132, and the energy output adjustment section 133.

The image acquisition section 131 acquires image data of the endoscope image received from the I/O device 180, and inputs the image data to the processor 110.

The tissue information recognition section 132 recognizes tissue information regarding the treatment target tissue from the endoscope image. The tissue information recognition section 132, for example, uses the trained model obtained by machine learning to recognize the tissue information. The tissue information may be various kinds of information. For example, the tissue information is a type of the treatment target tissue, a type of the energy treatment tool 320, contents of the treatment, or a scene. Alternatively, the tissue information is a state of the treatment target tissue regarding the energy treatment tool, which has been described with reference to the first embodiment or the like. The state is, for example, a wet state, or a result of evaluation of an applied tension. Alternatively, the tissue information is a result of evaluation of the risk for a thermal burn due to the proximity metal, which has been described with reference to the third embodiment.

For example, in a case where the energy treatment tool 320 is the hook-knife type monopolar device as described in the first embodiment and the tissue information includes a result of estimation of the tension in the treatment target tissue pulled by the hook, the tissue information recognition section 132 may include the first measurement section 112 and the second measurement section 113 similar to those in the first embodiment. Additionally, the first measurement section 112 may recognize the image of the monopolar treatment tool from the endoscope image and set the vicinity of the distal end of the monopolar treatment tool as the region of interest, and the second measurement section 113 may evaluate the tension in the treatment target tissue from the image of the region of interest. However, the energy treatment tool 320 may be an energy device of various kinds as described above, and the tissue information may include various kinds of information.

The energy output adjustment section 133 makes the output setting of the generator 300 based on the recognized tissue information. The energy output adjustment section 133 sets recommended output based on the tissue information, and transmits the recommended output to the generator 300 via the I/O device 170. For example, in a case where the tissue is a tissue on which incision is easily performed such as a thin membrane, a case where a tension is higher than the appropriate range, or a case where the risk for a thermal burn is higher than a predetermined value due to the metal or wetting, the energy output adjustment section 133 sets a recommended output level that is lower than normal output, or sets a recommended output sequence in which incision capability is lower than that of the normal output. In a case where the tissue is a tissue that requires sealing performance such as a blood vessel, or a case where a tension is lower than the appropriate range, the energy output adjustment section 133 sets a recommended output level that is higher than the normal output, or sets a recommended output sequence in which incision capability is higher than that of the normal output. Alternatively, the energy output adjustment section 133 may set, based on a detected scene, appropriate recommended output used in the scene. The energy output adjustment section 133 may refer to a table to convert the tissue information to a recommended setting, or may use a trained model obtained by machine learning to output the recommended setting. In a case of using machine learning, the tissue information recognition section 132 and the energy output adjustment section 133 may be implemented as an integrated trained model.

The output setting transmission section 139 notifies the operator of contents of the recommended output. Examples of the notification include monitor display, a sound, and vibrations. Examples of the monitor display include characters, figures, colors, and icons. The monitor display may be performed on the monitor 230 of the endoscope, or may be performed on a monitor that is not shown and that is separately provided to display various kinds of support information.

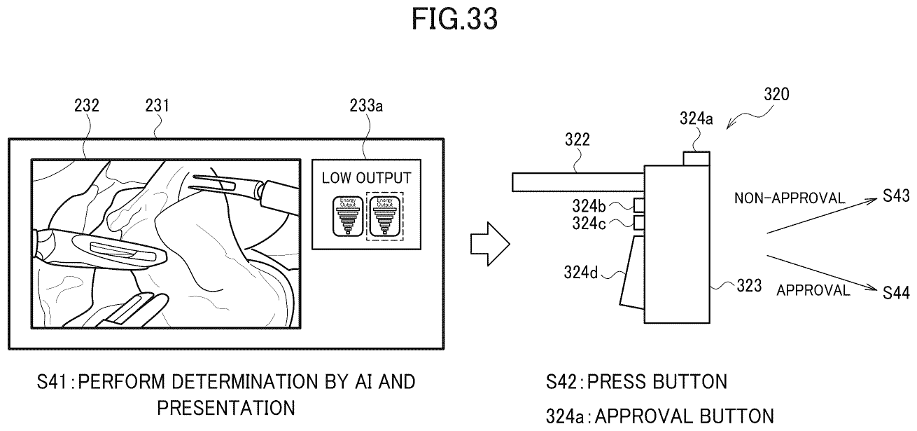

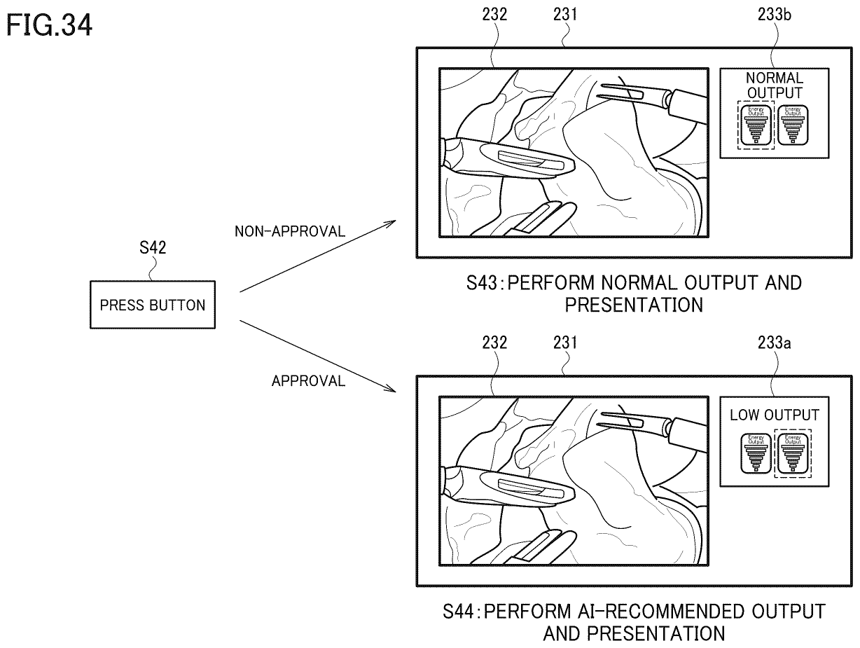

FIGS. 33 and 34 are flowcharts of a first detailed example of the fourth embodiment. An example of presenting the recommended setting with icons and characters on the monitor will be described below. The following description will be given of an example in which the energy treatment tool 320 grips the tissue with two jaws and performs energy output. Examples of such an energy treatment tool 320 include a bipolar device and a high-frequency/ultrasound combination device.

In step S41, the tissue information recognition section 132 and the energy output adjustment section 133 make determination about the recommended output, and the output setting transmission section 139 displays presentation information 233a of the recommended output on a monitor screen 231. The presentation information 233a is, for example, displayed together with an endoscope image 232 on the monitor screen 231.

As a timing for presenting the presentation information 233a, for example, there are mainly three conceivable patterns: (1) a timing before the tissue is gripped with the treatment tool; (2) a timing immediately after the tissue is gripped with the treatment tool; and (3) a timing after the elapse of predetermined time since the tissue is gripped with the treatment tool. At the timing of (1), the output setting transmission section 139 presents information when making determination about an intention to perform a treatment. For example, in machine learning, the training model has been trained with the endoscope image, together with a label indicating whether or not the tension is adequate or/and the distal end of the treatment tool is in proximity to the tissue, or whether or not the jaws make a transition from an open state to a closed state. The processor 110 uses the trained model to determine, based on the movement of the energy treatment tool 320 or the forceps for gripping in a surgical field, whether the purpose of the movement is merely an operation or an energy treatment. Note that the energy output adjustment section 133 may average results of determination about the recommended output so that contents of the presentation information 233a do not change frequently. Alternatively, the energy output adjustment section 133 may fix a result of determination about the recommended output unless the gripping state or the proximate state is released at any of the above-mentioned timings of (1) to (3).

As described in step S42, the energy treatment tool 320 includes a shaft and an end effector 322, a handle 323 that is gripped by the operator, a button 324a provided above the handle 323, and buttons 324a, 324c, and 324d provided on the front surface of the handle 323. It is assumed herein that the button 324a is an approval button and the button 324b is an output button, but which function is applied to any of the buttons may be freely determined. The arrangement of the buttons is not limited to that shown in FIG. 33.

In step S42, the operator approves or rejects the recommended output. Specifically, the operator presses an approval button 324a to approve the recommended output determined by the AI, and does not press the approval button 324a to reject the recommended output determined by the AI. The operator presses the output button 324b at a timing at which he/she wants to perform the energy treatment.

As described in step S43, in a case where the operator rejects the recommended setting, presentation information 233b indicating the normal output is displayed on the monitor. When the output button 324b is pressed, the generator 300 performs energy output with the normal setting. The normal setting is a predetermined output setting, a setting that is input from an operation portion of the generator 300 by the operator, or the like.

As described in step S44, in a case where the operator approves the recommended setting, the presentation information 233a indicating the recommended output is displayed on the monitor. When the output button 324b is pressed, the generator 300 performs energy output with the recommended setting.

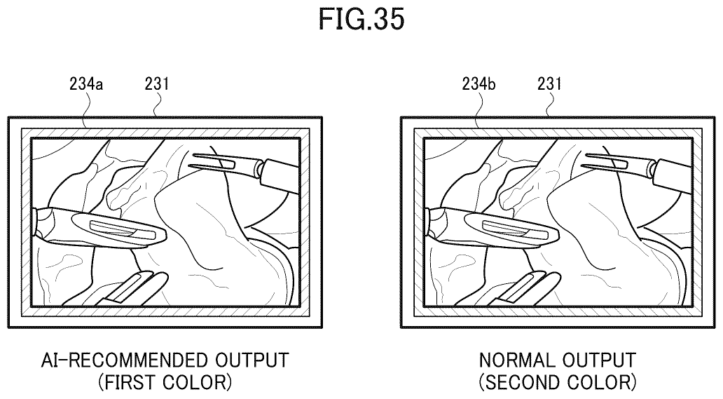

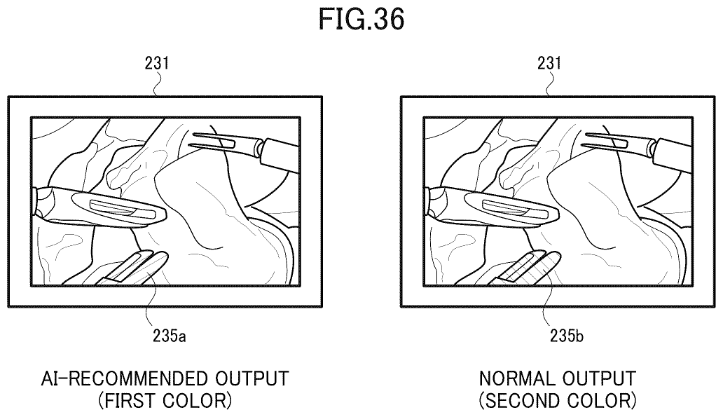

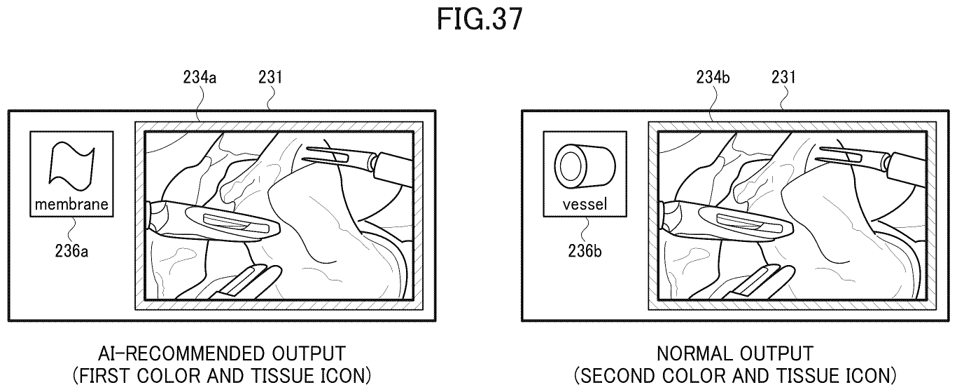

Various kinds of methods of presenting the presentation information can be assumed. For example, the following five examples are conceivable.
(1) As described with reference to FIGS. 33 and 34, the energy setting may be displayed in a location of the monitor screen 231 where the energy setting does not overlap with the endoscope image 232.
(2) As shown in FIG. 35, a frame in a color corresponding to the energy setting may be displayed around the endoscope image on the monitor screen 231. For example, a frame 234a in a first color is displayed in a case of the recommended output, and a frame 234b in a second color that is different from the first color is displayed in a case of the normal output. For example, the first color is blue, and the second color is orange.
(3) As shown in FIG. 36, the color corresponding to the energy setting may be superimposed on the energy treatment tool or in the vicinity of the energy treatment tool in the endoscope image. FIG. 36 shows an example in which the color is superimposed on the energy treatment tool. For example, display by superimposition 235a in the first color is performed in the case of the recommended output, and display by superimposition 235b in the second color that is different from the first color is performed in the case of the normal output. For example, the first color is blue, and the second color is orange.
(4) As shown in FIG. 37, an icon indicating what kind of a tissue is to be treated with the energy setting may be presented. As one example, shown is an example in which the normal output is the energy setting for treating a blood vessel, and the energy output adjustment section 133 recommends the energy setting for treating a thin membrane. As shown in the left drawing of FIG. 37, in a case where the operator approves the recommended setting, an icon 236a indicating a thin membrane is displayed around the endoscope image on the monitor screen 231. As shown in the right drawing of FIG. 37, in a case where the operator rejects the recommended setting, an icon 236b indicating a blood vessel is displayed around the endoscope image on the monitor screen 231. Note that FIG. 37 shows the example in which the display of the icon is combined with the display of the energy setting described in (2), but the display of the icon may be combined with the display of the energy setting described in (1) or (3).
(5) A sound or a voice corresponding to the energy setting may be emitted from a speaker or the like. For example, a low-frequency sound is emitted in the case of the recommended output, and a high-frequency sound is emitted in the case of the normal output. This allows the operator to recognize the change of the energy setting without taking his/her eyes away from the endoscope image.

In addition, the following examples are conceivable as modifications of the presentation method.

### (First Modification) Variations of the Information Display Position

(1a) When the color is displayed on the edge of the image, the color may be displayed, not on the whole perimeter, but on only one side. By arranging the color at 3° away in paracentral vision and within 20° in the region of interest, it is possible to give a heads-up without hindering a manipulation.

(1b) The presentation information may be displayed in the vicinity of the region of interest. By approximating the display to the paracentral vision, even the operator who tends to have central vision can recognize the presentation information. Visibility may be increased by making the color or the position more notable with distance from the region of interest.

### (Second Modification) Variations of the Notification Method

(2a) The energy setting may be presented with an icon. The operator can understand the energy setting with an image corresponding to settings on the generator side, sealing capability, incision speed, or ease of heat diffusion.

(2b) A tissue type or a result of evaluation of a state may be presented with an icon. The operator recognizes a gripped object with the tissue type such as a membrane and a blood vessel or with a recognizable state. Since the operator is presented with recognized contents themselves and there is no need for converting the recognized contents into the energy setting, the operator can understand the recognized contents with a straightforward image.

(2c) The notification method may be changed between a standby state for approval and during output. For example, in the standby state for approval, the energy setting is presented to the operator by display, (intermittent) blinking of light, the rotation of display, intermittent sounds, or intermittent vibrations. During the output, the energy setting is presented to the operator by display, blinking of light, translucent display, fixed display, a continuous sound, or the stop of vibrations. It becomes easier for the operator to recognize whether or not it is in a state where energy is output.

### (Third Modification) Combination of the Notification Methods

(3a) Display may be changed in synchronization with a sound or vibrations. It is known that stimuli from a plurality of sense organs increase reactivity, and synchronization of the display with the sound or the vibrations makes it easier for the operator to recognize the energy setting.

### (Fourth Modification) Variations of Blinking

(4a) A cycle of blinking or a duty ratio may be changed depending on the energy setting. Examples of a conceivable interval between ON and OFF of light emission in the blinking or between ON and OFF of light emission in the rotation include 0.15 to 0.4 s per cycle with a duty ratio of ON of light emission to OFF of light emission being 30 to 90%. Assuming that determination is prompted with an interval of about 0.5 s, only time corresponding to two blinks is left as recognition time with the cycle of 0.25 s. Thus, the number of blinks is hard to be further reduced. In addition, if the duty ratio of ON of light emission per cycle is small, black is notable for the human eyes, so that a color or blinking is hard to recognize.

(4b) An interval of blinking may be extended. The interval of blinking may become longer over time. If blinking time is changed to time favorable for treatment speed, the operator's concentration does not drop.

The following variations of the configuration of the approval input section 340 are conceivable.

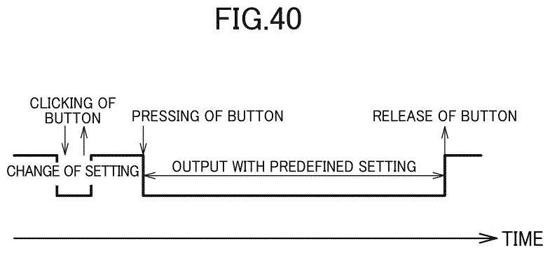

(1) (1a) As described with reference to FIG. 33, the energy treatment tool 320 is provided with the approval button other than the output button. For example, the output button is provided at a position so as to be pressed by an index finger, and the approval button is provided at a position so as to be pressed by a thumb. (1b) The approval button that is provided separately from the energy treatment tool 320 may be attachable to the energy treatment tool 320 with an attachment. An attachment position is, for example, on an upper surface of the handle. (1c) The approval button may be provided on a side surface of the handle. In accordance with 1a to 1c, the operator operates the approval button with a finger different from a finger to operate the output button. This allows the operator to recognize that the output is performed with a setting different from an existing energy setting. (1d) As described later with reference to FIG. 40, the output button may be used also as the approval button. In a case where the operator performs a click operation on the button, the operation serves as an operation for rejection. When the operator presses the button immediately after the click operation, the normal output is performed. In a case where the operator presses the button without performing the click operation, the operation serves as an operation for approval, and the output is performed with the recommended setting. Details thereof will be described later with reference to FIG. 40.

(2) An approval function may be allocated to a foot switch. The operator performs the operation for approval with a part different from the hand to operate the energy treatment tool 320. This allows the operator to recognize that the output is performed with a setting different from the existing energy setting.

A second detailed example of the fourth embodiment is now described. In this example, a method of transmitting the operator's rejection of the change of the energy setting is implemented. That is, in a case of rejecting a result of determination made by the system and a result automatically changed by the system, the operator presses a mechanism for inputting the rejection. The treatment can be performed with an operational feeling that is not different from that of the existing treatment tool. Since it is also possible to perform a treatment with the normal output when the operator cannot trust the results, the operator can freely select a treatment that the operator thinks is safe.

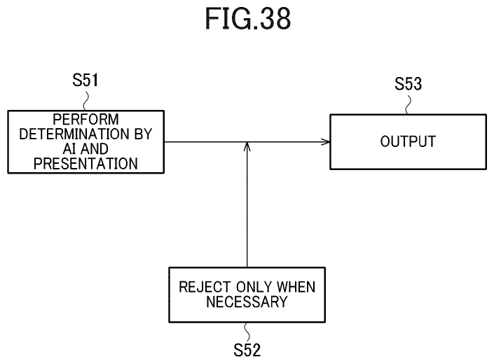

FIG. 38 is a flowchart of a second detailed example of the fourth embodiment. In step S51, the tissue information recognition section 132 and the energy output adjustment section 133 determine recommended output, and the output setting transmission section 139 presents the recommended output to the operator.

In step S52, the operator performs the operation for rejection only when it is necessary to reject the recommended setting. To approve the recommended setting, the operator does not perform the operation for approval or the operation for rejection.

In step S53, the operator presses the output button, and the generator 300 performs energy output. In a case where the recommended setting is rejected, the normal output is performed. In a case where the recommended setting is not rejected, the output is performed with the recommended setting.

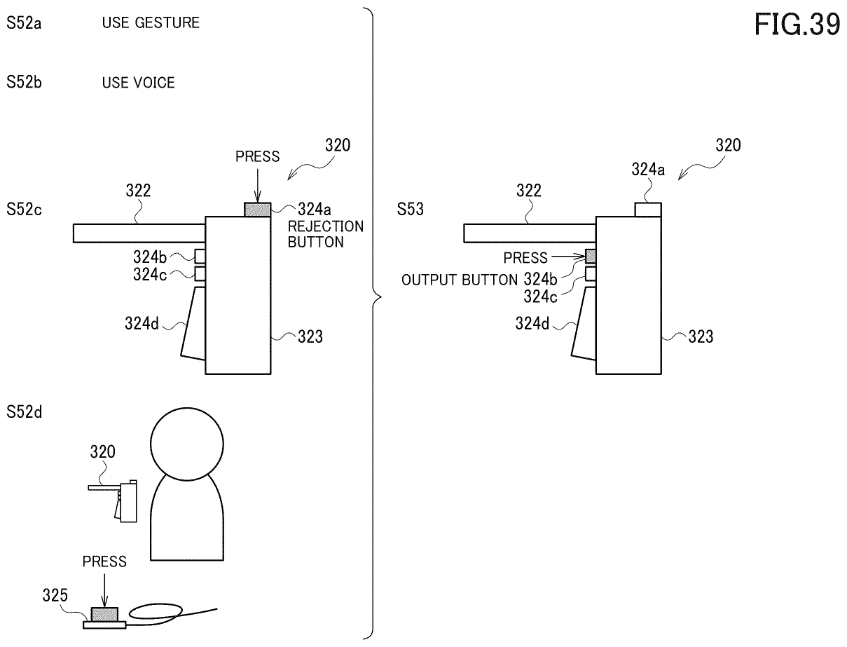

FIG. 39 shows a detailed example of steps S52 and S53.

Step S52a is an example of notifying the system of an intention of rejection with the operator's gesture.

Step S52b is an example of notifying the system of an intention of rejection with input of a voice via a microphone or the like.

Step S52c is an example in which a rejection button is provided in the energy treatment tool 320. The description herein is given of an example in which the button 324a on the upper surface of the handle 323 is the rejection button. To reject the recommended setting, the operator presses the rejection button 324a. To approve the recommended setting, the operator does not press the rejection button 324a.

Step S52d is an example in which the function of the rejection button is allocated to a foot switch 325.

In step S53, when the operator presses the output button 324b, the energy output is performed. The description herein is given of an example in which the output button is allocated to the button 324b at a position so as to be operated with the index finger when the operator grips the handle 323.

FIG. 40 shows an example in which the function of approval or rejection and the function of energy output are allocated to one button. The button may be a button provided in the energy treatment tool 320 or may be a foot switch.

As shown in FIG. 40, to reject the recommended setting, the operator performs a click operation on the button, and thereafter presses the identical button. While the button is being pressed, the energy output is performed with the normal setting. To approve the recommended setting, the operator presses the button without performing the click operation. While the button is being pressed, the energy output is performed with the recommended setting. When the energy output is performed with the recommended setting, to change the energy setting to the normal setting, the operator temporarily releases the button and thereafter presses the button. Thereafter, the energy output is performed with the normal setting while the button is being pressed. Note that there are some conceivable variations of a behavior of the change of the setting in a case where the button is continuously pressed. For example, in a case where a meaning of the operator's rejection of the recommended setting is given to the pressing of the button, the setting is changed to the normal setting also by the pressing of the button multiple times, and no further change is made. Alternatively, in a case where a meaning of the operator's rejection of the setting being currently presented is given, it is conceivable that the setting is cyclically changed in response to the pressing of the button multiple times.

The following variations of the timing for approval or rejection are conceivable.
(1) The determination is made each time: the operator makes determination about approval or rejection for every energy output. It is possible to confirm the operator's intention infallibly.
(2) The determination is made when the setting is changed and the determination is omitted when the setting is continuously used: the operator makes determination about approval or rejection only when the system changes the recommended setting, or the operator wants to change the setting. In the other states, the present setting is presented. The operator can always refer to the output setting, and the decrease of a frequency of determination decreases a burden on the operator.
(3) The determination is made when the setting is changed to the recommended setting: the operator makes determination about approval or rejection only when the operator changes the setting from the normal setting to the recommended setting. Since the operator knows effects of the existing setting and the determination is requested only in a case where the setting is changed to the recommended setting, the decrease of the frequency of determination decreases the burden on the operator.
(4) Turning ON of a function presented at the time of non-gripping in a surgery scene in which the function is expected to be effective: since it is possible to constantly perform surgery with the normal setting in most of a time zone of surgery time, energy is not changed. Turning ON the function for making the determination about the recommended output using the AI only in a scene in which the system can provide a medical value such as prevention of a thermal burn can further decrease the burden on the operator. There is the following variation (5) or (6) of the “surgery scene in which the function is expected to be effective”.
(5) In a case where a macroscopic surgery scene is obtained from the outside and an intention to use the AI-recommended output is presented: the AI-recommended output is automatically turned ON depending on a surgery scene. Since the recommended output is presented to the operator in a state where a treatment is not to be performed, the operator can preliminarily recognize the output setting and safely use the function.
(6) In a case where an important organ that has been a target of training is recognized within a field of view and an intention to use the AI-recommended output is presented: the AI-recommended output is automatically turned ON in a simpler manner at a time point when the important organ enters the field of view. Since the recommended output is presented to the operator in a state where a treatment is not to be performed, the operator can preliminarily recognize the output setting and safely use the function.

A third detailed example of the fourth embodiment is now described. In this example, different output sounds are allocated to respective energy settings. The system only emits only a beep sound without emitting energy for short amount time after the energy application button is pressed. The operation of the energy treatment is not different from that of the existing treatment tool. However, there is a time lag from the pressing of the energy application button to the application of energy, the operator can become aware of a difference from energy that is assumed by the operator during this time lag, and stop the energy output.

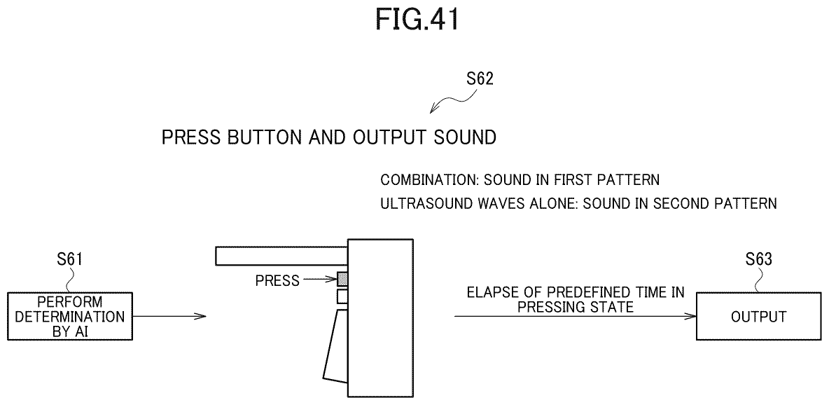

FIG. 41 is a flowchart of a third detailed example of the fourth embodiment. In step S61, the tissue information recognition section 132 and the energy output adjustment section 133 determine recommended output. The medical system internally changes the energy output setting without presenting a result of determination to the operator.

In step S62, when the operator presses the output button, the medical system emits an output sound corresponding to the recommended setting. After the output button is pressed, the energy output is not performed until after the elapse of predefined time. High-frequency wave/ultrasound wave combination device is taken as an example. When output of combining high-frequency waves and ultrasound waves is selected, the medical system outputs a sound in a first pattern. When output of ultrasound waves alone is selected, the medical system outputs a sound in a second pattern that is different from the first pattern. For example, the sound in the first pattern is a sound at a low frequency, and is, for example, an intermittent sound at about 250 Hz. The sound in the second pattern is a sound at a high frequency, and is, for example, an intermittent sound at about 500 Hz.

In step S63, the operator determines whether or not the recommended setting is appropriate during a period from the pressing of the button to the start of application of energy. When the predefined time elapses while the operator keeps pressing the output button, the medical system performs the energy output with the recommended setting. When the operator releases the output button before the elapse of the predefined time, the medical system does not perform the energy output.

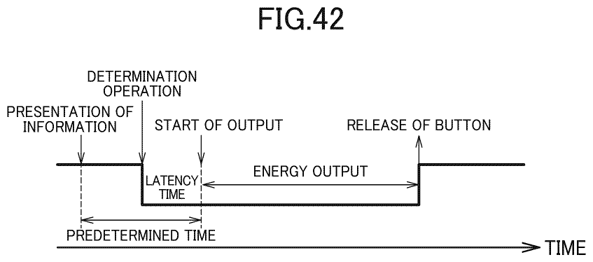

FIG. 42 shows an example of providing delay time starting, not from pressing of the button, but from presentation of information to the start of output. The tissue information recognition section 132 and the energy output adjustment section 133 determine the recommended output, and the output setting transmission section 139 presents the recommended output to the operator. When the operator presses the output button before the elapse of the predetermined time from the presentation of the information, the medical system waits for the elapse of the predetermined time from the presentation of the information and starts the energy output with the recommended setting. When the operator presses the output button after the elapse of the predetermined time from the presentation of the information, the medical system starts the energy output with the recommended setting at a timing when the output button is pressed. The predetermined time from the presentation of the information to the start of the output is preferably 0.1 to 0.8 s, more preferably about 0.3 s. In a case where the operation for rejection is input and the operator's intention is trustworthy based on the operation for changing the setting, the delay time need not be provided.

The predetermined time is, for example, 100 ms or more. Response time to a simple stimulus is said to be 150 to 300 ms. There is a possibility that the response time becomes about 100 ms when training is performed, and a false start in athletic sports is set at about 100 ms. That is, a response in a time shorter than the response time cannot be said as an action after the presentation is recognized. Alternatively, the predetermined time is 600 ms or less. A threshold of simultaneousness is said to be 150 to 300 ms. Many people feel a delay with respect to time longer than the threshold. Thus, when the time is delayed for the longest time of the threshold of the simultaneousness of 300 ms or more from the longest time of the response time to the simple stimulus of 300 ms, there is a possibility that the time becomes a time with which a considerable number of people become conscious of a delay with respect to the operation, and usability decreases.

A fourth detailed example of the fourth embodiment is now described. In this example, the medical system performs scene recognition, and recommends a basic setting that is appropriate for a main operation in a scene. The operator gives approval in a case where the presentation is appropriate in the scene. The system makes a request for approval to the operator only when a tissue that is different from the target tissue of the main operation is gripped in the treatment. If the operator gives approval, the energy setting is changed to the recommended setting, and the operator performs the treatment. By making the energy setting suitable for the main tissue or the state, the AI-recommended setting is not changed until the main tissue or the state changes. With this configuration, it is possible to reduce the number of the operations for approval itself, the operations being performed by the operator to give approval to the energy change.

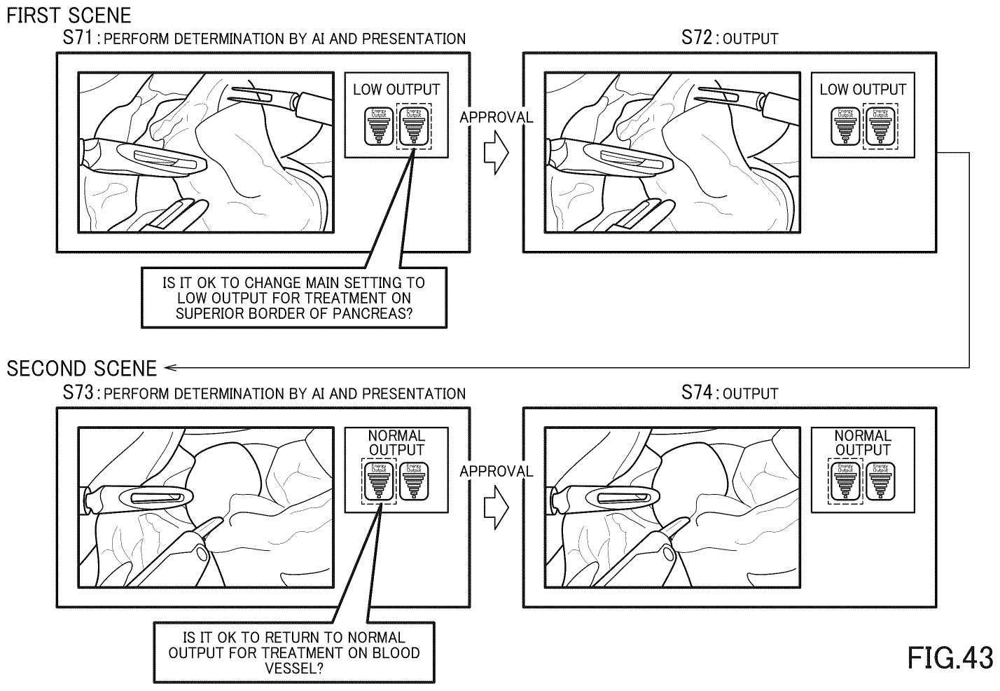

FIG. 43 is a flowchart of a fourth detailed example of the fourth embodiment. In step S71, the tissue information recognition section 132 and the energy output adjustment section 133 recognize a scene and determine recommended output in the scene. The output setting transmission section 139 presents the recommended output to the operator. Audio guidance may be performed together with the image display Assume that the operator has approved or has not rejected the presentation. In step S72, when the operator presses the output button, energy is output with the recommended setting. The medical system neither changes the recommended setting nor makes a request for the operation for approval or rejection until recognizing that the scene has been changed to a new scene. When the output button is pressed, the medical system performs energy output with the recommended setting. For example, in a case where a first scene is a scene to treat a thin membrane, the medical system neither changes the recommended setting nor makes a request for the operation for approval or rejection while recognizing that the gripped tissue is a thin membrane.

In step S73, the tissue information recognition section 132 and the energy output adjustment section 133 recognize a new scene and determine recommended output in the scene. The description herein is given of an example in which the normal output is recommended with respect to a second scene. The output setting transmission section 139 presents the normal output to the operator. Audio guidance may be performed together with the image display. Assume that the operator has approved or has not rejected the presentation. In step S74, when the operator presses the output button, energy output is performed with the normal setting. For example, in a case where the second scene is a scene to treat a tissue other than a thin membrane such as a blood vessel, the medical system neither changes the normal setting nor makes a request for the operation for approval or rejection while recognizing that the gripped tissue is the tissue other than a thin membrane.

As described above, the medical system 10 in the present embodiment includes the treatment tool 310 provided with the end effector that treats the tissue, the generator 300 that adjusts energy output from the end effector, the imaging device that captures an image of the tissue, and the processor 110. The processor 110 outputs a recommended output setting of the generator 300 and accepts operation input to approve or reject the recommended output setting. When the recommended output setting is approved or not rejected in the operation input, the processor 110 performs third processing of changing the output setting of the generator 300 to the recommended output setting.

In a case where AI is introduced into the medical system, whether or not to adopt determination by the AI is preferably determined by the operator. The present embodiment allows the operator to approve or reject the AI-determined, recommended output setting of the generator 300. With this configuration, the function for approval or rejection enables a treatment with an output setting desired by the operator, while assisting the operator with use of the AI reduces a burden on the operator.

Additionally, in the present embodiment, the processor 110 may acquire the color information of the target tissue from the captured image output from the imaging device. The target tissue is a portion of the tissue that covers at least a portion of the end effector 311 that has slid into the tissue. The processor 110 may estimate a force applied to the target tissue by the end effector 311 from the color information of the target tissue. The processor 110 may change the output setting of the generator 300 based on a result of estimation of the force.

With this configuration, it is possible to estimate the force applied to the target tissue from the color information of the target tissue using a method similar to that in the first embodiment. The method can be applied to, for example, a case where the treatment tool 310 is the hook-knife type monopolar device or other cases.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

1. A medical system comprising:
a treatment tool provided with an end effector that treats a tissue;
an imager capturing an image of the tissue;
a processor; and
a memory including a program,
the memory and the program being configured to, together with the processor, cause the medical system to:
acquire color information of a target tissue from the captured image output from the imager, the target tissue being a portion of the tissue that covers at least a portion of the end effector that has slid into the tissue;
estimate a force applied to the target tissue by the end effector from the color information of the target tissue; and
notify an operator of a result of the estimation of the force.

2. The medical system as defined in claim 1, wherein
the memory and the program, together with the processor, cause the medical system to:
recognize a position of the treatment tool in the captured image;
set a region of interest to a region in the captured image corresponding to the end effector based on a result of the recognition of the position;
recognize the color information of the target tissue from an image of the region of interest; and
estimate the force from the color information of the target tissue.

3. The medical system as defined in claim 2, wherein
the treatment tool includes a shaft connected to a base end of the end effector, and
the memory and the program, together with the processor, cause the medical system to:
recognize at least one of the end effector or the shaft from the captured image by segmentation; and
set the region of interest by using a result of the recognition through the segmentation.

4. The medical system as defined in claim 3, wherein the memory and the program, together with the processor, cause the medical system to set a region that is surrounded by edge extension lines extended from respective edges of the shaft recognized by the segmentation and that includes the end effector as the region of interest.

5. The medical system as defined in claim 4, wherein
the memory and the program, together with the processor, cause the medical system to:
set a region surrounded by the edge extension lines and first and second perpendicular lines that are perpendicular to a principal component long-axis vector of the shaft as the region of interest;
the first perpendicular line is a line passing through the base end of the end effector or a line passing through a distal end of the shaft; and
the second perpendicular line is a line passing through a distal end of the end effector.

6. The medical system as defined in claim 4, wherein
the memory and the program, together with the processor, cause the medical system to:
set a region surrounded by the edge extension lines and first and second perpendicular lines that are perpendicular to a principal component long-axis vector of the shaft as the region of interest;
the first perpendicular line is a line passing through a distal end of the shaft; and
the second perpendicular line is a line away from the first perpendicular line by a predetermined length toward a distal end side of the end effector.

7. The medical system as defined in claim 3, wherein
the memory and the program, together with the processor, cause the medical system to:
recognize a missing region of the end effector that is missing in the captured image due to overlapping of the end effector with the target tissue and an exposure region of the end effector seen in the captured image by the segmentation; and
set the recognized missing region and the recognized exposure region as the region of interest.

8. The medical system as defined in claim 1, wherein the memory and the program, together with the processor, cause the medical system to estimate that the force applied to the target tissue by the end effector becomes stronger as the color information of the target tissue becomes closer to color information of the end effector.

9. The medical system as defined in claim 8, wherein
the memory and the program, together with the processor, cause the medical system to:
acquire transmittance of a color of the end effector in the target tissue as an index indicating approximation between the color information of the target tissue and the color information of the end effector; and
estimate that the force applied to the target tissue by the end effector becomes stronger as the transmittance becomes higher.

10. The medical system as defined in claim 8, wherein the memory and the program, together with the processor, cause the medical system to make a comparison between an index indicating approximation between the color information of the target tissue and the color information of the end effector and a determination criterion for determination about the index indicating an appropriate force range to determine whether or not the force applied to the target tissue is in an appropriate range.

11. The medical system as defined in claim 10, wherein
the memory and the program, together with the processor, cause the medical system to:
recognize a type of the target tissue from the captured image;
select a determination criterion corresponding to the recognized type from a plurality of determination criteria corresponding to a plurality of tissue types; and
determine whether or not the force applied to the target tissue is in the appropriate range using the selected determination criterion.

12. A medical system comprising:
a treatment tool provided with an end effector that treats a tissue;
a generator that adjusts energy output from the end effector;
an imager capturing an image of the tissue;
a processor; and
a memory including a program,
the memory and the program being configured to, together with the processor, cause the medical system to:
perform first processing of estimating a force applied to the tissue to be treated by the treatment tool from the image captured by the imager and changing an output setting of the generator based on a result of the estimation of the force;
perform second processing of detecting a metal in a periphery of the end effector from the image captured by the imager and changing the output setting of the generator based on a result of the detection of the metal, or
perform third processing of outputting a recommended output setting of the generator, accepting operation input to approve or reject the recommended output setting, and, in a case where the recommended output setting is approved or not rejected in the operation input, changing the output setting of the generator to the recommended output setting; and
wherein, in a case where the first processing or the third processing is performed,
the memory and the program, together with the processor, cause the medical system to:
acquire color information of a target tissue from the captured image output from the imager, the target tissue being a portion of the tissue that covers at least a portion of the end effector that has slid into the tissue;
estimate the force applied to the target tissue by the end effector from the color information of the target tissue; and
change the output setting of the generator based on a result of the estimation of the force.

13. The medical system as defined in claim 12, wherein
the memory and the program, together with the processor, cause the medical system to:
recognize a position of the treatment tool in the captured image;
set a region of interest to a region in the captured image corresponding to the end effector based on a result of the recognition of the position;
recognize the color information of the target tissue from an image of the region of interest; and
estimate the force from the color information of the target tissue.

14. The medical system as defined in claim 13, wherein
the treatment tool includes a shaft connected to a base end of the end effector, and
the memory and the program, together with the processor, cause the medical system to:
recognize at least one of the end effector or the shaft from the captured image by segmentation; and
set the region of interest by using a result of the recognition through the segmentation.

15. The medical system as defined in claim 14, wherein
the region of interest is a range in which the end effector should be seen in the captured image, and
the memory and the program, together with the processor, cause the medical system to set a region that is surrounded by edge extension lines extended from respective edges of the shaft recognized by the segmentation and that includes the end effector as the region of interest.

16. The medical system as defined in claim 12, wherein the memory and the program, together with the processor, cause the medical system to estimate that the force applied to the target tissue by the end effector becomes stronger as the color information of the target tissue becomes closer to color information of the end effector.

17. The medical system as defined in claim 16, wherein
the memory and the program, together with the processor, cause the medical system to:
acquire transmittance of a color of the end effector in the target tissue as an index indicating approximation between the color information of the target tissue and the color information of the end effector; and
estimate that the force applied to the target tissue by the end effector becomes stronger as the transmittance becomes higher.

18. A notification method comprising:
acquiring color information of a target tissue from a captured image output from an imager capturing an image of a tissue, the target tissue being a portion of the tissue that covers at least a portion of an end effector of a treatment tool that has slid into the tissue;
estimating a force applied to the target tissue by the end effector from the color information of the target tissue; and
notifying an operator of a result of the estimation of the force.
